(19) United States
(12) Patent Application Publication
(10) Pub. No.: US 2022/0277852 A1
(43) Pub. Date: Sep. 1, 2022

(54) OPTIMIZING AUTONOMOUS SELF USING NON-INVASIVE MEASUREMENT SYSTEMS AND METHODS

(71) Applicant: HI LLC, Los Angeles, CA (US)

(72) Inventors: Bryan Johnson, Culver City, CA (US); Julian Kates-Harbeck, Marina Del Rey, CA (US); Ryan Field, Culver City, CA (US); Patrick House, Seattle, WA (US); Katherine Perdue, Los Angeles, CA (US)

(73) Assignee: HI LLC, Los Angeles, CA (US)

(21) Appl. No.: 17/592,838

(22) Filed: Feb. 4, 2022

Related U.S. Application Data
(60) Provisional application No. 63/154,123, filed on Feb. 26, 2021.
(60) Provisional application No. 63/179,957, filed on Apr. 26, 2021.
(60) Provisional application No. 63/160,766, filed on Mar. 13, 2021.
(60) Provisional application No. 63/173,341, filed on Apr. 9, 2021.
(60) Provisional application No. 63/235,039, filed on Aug. 19, 2021.

(51) Int. Cl. G16H 50/30; G16H 50/20; G16H 10/60; A61B 5/00

(52) U.S. Cl. CPC G16H 50/30; G16H 50/20; G16H 10/60; A61B 5/0022; A61B 5/4806; A61B 5/6803; A61B 5/6814

(57) ABSTRACT

A non-invasive self-autonomous system and method of optimizing a lifestyle regimen of a person containing a combination of lifestyle variables is provided. At least one value of the combination of lifestyle variables is repeatedly modified, thereby creating different variations of the combination of lifestyle variables respectively having different sets of values. The different variations of the combination of lifestyle variables are sequentially administered to the person. Physiological activity of the person is detected in response to the administration of the combination of lifestyle variables to the person. Sets of qualitative indicators of an aspect of a lifestyle of the person are derived from the detected physiological activity of the person. The lifestyle regimen of the person is optimized based on the different variations of the combination of lifestyle variables and the derived sets of qualitative indicators.

### RELATED APPLICATION DATA

Pursuant to 35 U.S.C. § 119(e), this application claims the benefit of U.S. Provisional Application Ser. No. 63/154,123, filed Feb. 26, 2021, U.S. Provisional Application Ser. No. 63/179,957, filed Apr. 26, 2021, U.S. Provisional Application Ser. No. 63/160,766, filed Mar. 13, 2021, U.S. Provisional Application Ser. No. 63/173,341, filed Apr. 9, 2021, and U.S. Provisional Application Ser. No. 63/235,039, filed Aug. 19, 2021, which are all expressly incorporated herein by reference.

### FIELD OF THE INVENTION

The present inventions relate to methods and systems for non-invasive measurements in the human body for modulating the lifestyle of a human.

### BACKGROUND OF THE INVENTION

Our fixed biological energy capacity is a fundamental constraint to what we can do or become in life. To overcome or make optimal use of this limit, we use technology to expand and extend our abilities. Improvements in biological energy expenditure underlie humanity's progress, e.g., hand stitch to loom, wagons to horses to the combustion engine, abacus to computers, etc. If we want to think really big about the future of being human, we need more advanced ways of managing our biological energy expenditure. Much like fire freed our ancestors from certain caloric and dietary restrictions, which opened up energy—i.e., metabolism/time—allowing functions like language and society as we know it to develop, achieving peak performance and improving ourselves continuously in an autonomous manner (the “Autonomous Self”) will free up just as much energy, allowing humans to explore new frontiers, e.g., developing new industries, discovering original uses of the mind, making iterations of governance and economics, and exploring the goal alignment problem within ourselves, between each other, and with artificial intelligence (AI).

The Autonomous Self is based on the premise that it is more than our conscious awareness and the symbolic terms and ontological primitives we have to represent it today. Two examples of ontological primitives may be sleep and biomarkers that we do not necessarily consider to be part of “Self,” largely because each is also simultaneously self-directed and autonomous, with very little cognitive control over when or why our bodies crave the things they do. Fighting to stay awake, as we all know, is a losing battle.

Our subconscious and continuous dynamic interaction with the outside world can be thought as an extended, expansive Self that has gradations of being autonomous (e.g., digestion, wound healing and driving, etc.) and self-directed (e.g., voluntary control such as problem solving). The Autonomous Self may be considered to have six levels. One way to think about the Autonomous Self is through a familiar lens of autonomous airplanes or cars. Most of what we do, most of what we think, and most of what our body processes and manages, is done entirely without our knowledge or awareness.

At level 0 (no automation), the automated system issues warnings and may momentarily intervene, but has no sustained bodily or cognitive control. The Autonomous Self is in charge of full-time performance of all living tasks, even if “enhanced by warning or intervention systems.” Examples of level 0 automation with respect to cars may include, e.g., a seat belt alarm, dashboard warnings, collision/swerve detection, etc., whereas examples of level 0 automation with respect to body/cognition may include, e.g., light versus dark detection, sound versus no sound detection, odor versus no odor detection, etc.

At level 1 (assistance), the automated system shares control by using information about the environment, with the expectation that the Autonomous Self performs all remaining aspects of the task. Examples of level 1 automation with respect to cars may include, e.g., cruise control, adaptive braking, parking assistance, etc., whereas examples of level 1 automation with respect to body/cognition may include phototropy, chemotropy, pupil response, fight or flight response, regulation of respiratory rate and pulse, thermoregulation, etc.

At level 2 (partial automation), the automated system takes full control of movement and cognitive basics. The Autonomous Self must monitor and be prepared to intervene immediately at any time if the automated system fails to response properly. Examples of level 2 automation with respect to cars may include, e.g., car has full control of accelerating, braking, steering, etc., but driver must keep watch and hands on the steering wheel at all times and driver performs all other activities with respect to control of the car, whereas examples of level 2 automation with respect to body/cognition may include, e.g., wound response/repair where Self is expected to use pain signals to avoid further damage or use, metabolic/dietary cravings generated by the automated system where Self is expected to seek out necessary food/nutrients.

At level 3 (conditional automation), the Autonomous Self can safely turn their attention away from most cognitive tasks with the assumption that it will and can intervene when requested. Examples of level 3 automation with respect to cars may include, e.g., driver must be vigilant and physically prepared for emergencies, rain, parking lots, etc., but can mostly turn attention elsewhere, whereas examples of level 3 automation with respect to body/cognition may include, e.g., skilled/learned movements, walking, language generation, etc.

Level 4 (high automation) is similar to level 3, but attention is recommended, but not required for safety, optimality, or health. The Autonomous Self will fix errors if it responds improperly. Examples of level 4 automation with respect to cars may include, e.g., robotic taxi or delivery service that does not need human intervention, car has the ability to stop itself, etc., whereas examples of level 4 automation with respect to body/cognition may include digestive system, all of perception, etc.

At level 5 (full automation), no intervention is required at all. The Autonomous Self goes about one's day optimally guided for all the operational cognitive tasks and decisions that are through undeserving of attention, freeing up metabolic capacity to explore higher order cognitive discovery. There are no examples of level 5 automation with respect to cars or body/cognition.

It would be desirable to optimize the level 5 Autonomous Self by converting self-directed activities to run autonomously, and for such autonomous activities to improve continuously without attention from the Autonomous Self. For example, there is compelling evidence that getting a good night's sleep not only improves one's physical and mental health (e.g., reducing obesity, Type 2 Diabetes, cardiovascular disease, cancer, dementia, depression, etc.), an aspect of impulse control during any particular day is determined by the amount of deep and total sleep the night before. That is, the more quality of sleep a person has, the better impulse control that person has (see Bryan Johnson, “Sleep and Impulse Control” (https://www.kernel.com/news/sleep-and-impulse-control; Brett Ryder, “Sleepless in Silicon Valley,” The Economist, May 16, 2019 (https://www.economist.com/business/2019/05/16/sleepless-in-silicon-valley).

Although improvements in a person's health, including impulse control, can be theoretically achieved through a good night's sleep, due to practical countervailing factors, actually getting a good night's sleep may not always be achieved. For example, what is one willing to change for a good night's sleep? Conversely, what is one willing to give up to avoid having a bad night's sleep? Up until now, answering this kind of question in a way that clearly justifies these sacrifices has been out of reach, forcing us to rely on guesswork. The lack of data on associated costs has resulted in our cultural normal devaluing and deprioritizing sleep. For example, when was the last time your primary care doctor asked to see your sleep performance data? Thus, it would be desirable to generate a sleep quality regimen for a person in a manner that the amount of biological energy expended by the person in generating and maintaining the sleep quality regimen is minimized.

Based on conscious insight by a person, some elements of their sleep quality regimen can be automated through habit formation or permanent physical set ups. For example, circadian rhythms are genetically encoded and enabled by a neural pacemaker in each person, and such person may have hunches about whether they are morning or night people or whether they have slept a bit too little or too much the night before, such that their conscious mind may effortlessly perform approximation of their sleep quality regimen. Other factors may be well-known to improve (or have a deleterious effect on) the quality of sleep, e.g., not eating right before bedtime, utilizing blue light blocking glasses and deep-wave sound machine utilized, sleeping in a temperature-controlled room, etc. Thus, these factors may be implemented into a sleep quality regimen without much thought. However, other elements of the sleep quality regimen may be variable, and thus, may require real-time management. For example, what, when, and how much one eats depends on their exercise routine for the day may need to be managed. These variables may affect the resting heart rate and heart rate variability (HRV) of a person, which are strongly correlated with high quality sleep. Other variables, such as caffeine intake, light exposure, supplements, bedtime routines (e.g., watching movie versus reading book), HRV training, meditation, etc., may differently affect the sleep quality of persons, and are therefore, individual dependent. Keeping this information at the top of one's mind, while trying to be functional in other aspects of life, is a challenging, yet critical, variable.

One can theoretically focus on a few of these variables and conduct hundreds of experiments in a quest to determine what lifestyle produces a perfect night of sleep. However, it would be challenging, if not impossible, for one to include a few dozen variables in each experiment to find new relationships between the variables in a quest to continually optimize one's health algorithm. Thus, manually building a health algorithm, and even more so continually updating that health algorithm, requires an enormous amount of time, attention, and dedication.

Related to a person's health is biological age, which in contrast to chronological age (i.e., the amount of time since birth), is the age that a person's body resembles or functions. As we age, the molecules, cells, tissues, and organs within our bodies undergo changes. The biology of the ageing process is complex, and is yet to be fully characterized. Though precise definitions of ageing can be controversial, it can generally be seen as the gradual accumulation of deleterious biological changes accompanying a progressive loss of function. It is clear, however, that ageing increases the risk of morbidity and mortality in humans (see James H. Cole, et al, “Brain Age and Other Bodily ‘Ages’: Implications for Neuropsychiatry,” Molecular Psychiatry (2019) 24: 266-281). It is also clear that humans do not experience biological ageing at the same rate, with pronounced differences in the outward manifestations of ageing being observed (e.g., hair loss, skin wrinkles, presbyopia) (see Id.). Thus, even though two people may both be thirty years old chronologically, one of them could have a biological profile that is closer to twenty-five, whereas the other might have a biological profile of thirty-five (see https://goop.com/wellness/health/what-is-my-biological-age/). Thus, biological age relative to chronological age is a better indicator of health span or the estimated length of a highly functional and disease-free life of a person.

In addition to physical manifestations, ageing has consequences for the brain and any patient suffering from chronic psychiatric or neurological disorder will be exposed to ageing effects during the course of their disease. Behaviorally, brain ageing is associated with cognitive decline (commonly described as cognitive ageing; particularly affecting cognitive domains, such as information processing speed, memory, reasoning, and executive functions (see James H. Cole, Ibid). The importance of maintaining a healthy brain during ageing is increasingly being recognized as a goal for society (Id.)

Unlike chronological age of a person, which cannot be changed, the biological age of the same person can be changed with health-related decisions. Certain activities, such as getting enough sleep and eating fruits and vegetables, are known to improve the biological age of a particular person. However, the concept of biological age is still fairly new, so it is not known specifically what activities and habits are the most ideal (e.g., how much exercise and what type of exercise, how much fasting or when to fast, type and dose of pharmaceutical drugs (or lack thereof), genetic manipulations, etc.), and such activities and habits may differ from person-to-person (see Id.).

In the same manner as it is difficult and time consuming to optimize one's health algorithm, as discussed above, it would be difficult and time consuming to continually optimize one's biological age by experimentation of a few dozen variables.

Although feeling great and decreasing one's biological age is alone worth the effort of autonomously improving the quality of one's sleep and health in general, the greatest interest in the Autonomous Self is to determine a path to the future of being human. The Autonomous Self can be further understood through the frame offered by Alfred North Whitehead, renown Mathematician and Philosopher, who once said “civilization advances by extending the number of important operations which we can perform without thinking about them.” Whitehead's point applies to human learning and growth too. Humans grow more autonomous by increasing the number of important operations that they can perform without thinking about them.

Thus, our future existence requires that we level ourselves up as a species, and at the fastest evolutionary speed in history. To do this, we need to free ourselves of the costly metabolic things we do today, such as rote or biased decision making and logistics management around solvable things, such as sleep and biomarker-based diet, exercise, or lifestyle. We can, therefore, level ourselves up to spend our precious time and energy to explore the frontiers of being human rather than things we know how to do efficiently. Thus, it is in the interest of civilization for us to make ourselves as autonomous as possible in the pursuit of making the best versions of ourselves, i.e., optimizing our behaviors.

Because it is difficult to organize every single aspect of our life, humans tend to focus only on those aspects that maximize improvement in our performance (e.g., we focus on the twenty percent of aspects in life that have an eighty percent benefit (the “80/20” rule)), thereby, in a sense, using our biological energy for the most important functions, long tail gains in performance in our lifestyle may be consciously ignored by us. For example, there may be five aspects that add up to a seven percent performance improvement, but this may not be taken advantage of because it is too much trouble.

Whether implementing an 80/20 rule or attempting to consciously manage every aspect of one's life, it is difficult to change what is not measured, and in this case, it is difficult to free up biological energy in the form of brain activity based merely on conscious intuition. Thus, if we could really measure our brain's activity in a normal life and work environment, it would be a historical turning point for humans, making radical human cognition evolution possible. It has hypothesized that brain activity may be quantified in terms of “attebytes” (see Bryan Johnson, “Changing Our Minds One Attebyte at a Time,” Apr. 19, 2018 (https://medium.com/future-literacy/changing-our-minds-one-attebyte-at-a-time-764692703636). Attebytes is not computational because the brain is not a CPU, and is not just a quantification of “attention,” because attention is only one small function of the brain (The root “attendere” means “stretching one's mind toward something.”). Rather, Attebytes may be simplistically thought as a number system that keeps track of what we spend our day thinking about, like a calorie counter for thoughts. Alternatively, Attebytes may be thought as an allocation of metabolism to brain regions of interest or selective bottlenecks of information processing over time.

Estimates are that each and every one of us experiences almost 80,000 thoughts a day. Each of these thoughts might be listed in a cognitive dashboard with a quantity of attebytes assigned to each thought, similar to how we measure, analyze, and optimize other systems, such as computers (i.e., CPU, Memory, Energy, Disk, Network, etc.), health (i.e., blood, vitals, genome, microbiome, etc.), climate/environment, automobiles, etc. For example, an exemplary cognitive dashboard for someone while getting dressed in the morning may look like:
Mentally rehearsing your 10 am presentation (8.3 attebytes)
Remembering that you wanted to change the title on slide #2 (0.56 attebytes)
Anticipating how you are going to emotionally respond when your coworker Miriam likely rejects the idea before even hearing you out (15.3 attebytes)
Realizing that your shoes looked terribly worn (0.40 attebytes)
Remembering that you have $767 in your bank account, but need to pay $2100 in rent next Tuesday to avoid to avoid the $75 late fee (5.3 attebytes)
Trying to decide whether to get new shoes (0.4 attebytes)
Wondering if the dilapidated state of your clothing reflects poorly on you (7.2 attebytes)

Some of these thoughts are not beneficial, and as such, it would be desirable to autonomously identify, quantify, and eliminate them) and the biological energy expenditure associated with them (i.e., via the Autonomous Self), so that we can become the best versions of ourselves, ultimately allowing humans to further evolve as a species.

### SUMMARY OF THE INVENTION

In accordance with one aspect of the present inventions, a non-invasive self-autonomous system is provided.

The non-invasive self-autonomous system comprises a peripheral device configured for administering a lifestyle regimen containing a combination of lifestyle variables to a user. In an optional embodiment, the peripheral device is configured for allowing the user to manually enter a value of a lifestyle variable, such that at least one variation of the combination of lifestyle variables has the manually entered value.

The non-invasive self-autonomous system further comprises at least one non-invasive measurement device configured for detecting physiological activity of the user in response to the administration of the lifestyle regimen to the user.

In one advantageous embodiment, the non-invasive measurement device(s) comprises a non-invasive brain interface assembly, the detected physiological activity of the user comprises detected brain activity of the user, and each derived set of qualitative indicators comprises a brain state of the user (e.g., a physiological brain state of the user and/or mental brain state of the user). The non-invasive brain interface assembly may be, e.g., an optical measurement assembly or a magnetic measurement assembly. In one embodiment, the invasive brain interface assembly comprises at least one detector configured for detecting energy from a brain of the user, and processing circuitry configured for identifying the brain activity in response to detecting the energy from the brain of the user. In this case, the non-invasive brain interface assembly may comprise a head-worn unit carrying the at least one energy source, and the non-invasive brain interface assembly may comprise an auxiliary non-head-worn unit carrying the processing circuitry.

In another embodiment, the non-invasive measurement device(s) comprises one or more peripheral sensors, and the detected physiological activity of the user comprises detected peripheral physiological activity (e.g., at least one of heart rate, heart rate variability, respiratory rate, blood pressure, blood flow, skin conductivity, blood glucose, Cortisol, DHEA-s, Serotonin, GABA, Dopamine, Norepinephrine, Epinephrine, and Glutamate).

The non-invasive self-autonomous system further comprises a lifestyle optimizer configured for modifying values of the combination of lifestyle variables of the lifestyle regimen, such that the peripheral device sequentially administers different variations of the combination of lifestyle variables respectively having different sets of values to the user. In one embodiment, the value of the lifestyle variable is currently performed by the user, in which case, the lifestyle optimizer may be configured for instructing the peripheral device to prompt the user to manually enter the currently performed value of the lifestyle variable. The lifestyle optimizer is further configured for deriving sets of qualitative indicators of an aspect of a lifestyle of the user from the detected physiological activity of the user respectively, and optimizing the lifestyle regimen of the user based on the different variations of the combination of lifestyle variables and the derived sets of qualitative indicators (e.g., using machine-learning). In one embodiment, the lifestyle optimizer is configured for selecting one of the different variations of the combination of lifestyle variables for the optimized lifestyle regimen.

In another embodiment, the lifestyle optimizer is configured for modifying the lifestyle regimen of the user, such that at least one of the derived sets of qualitative indicators substantially matches a target set of qualitative indicators. For example, the lifestyle optimizer may comprise a comparator configured for comparing each derived set of qualitative indicators and the target set of qualitative indicators and respectively generating at least one error signal, and a controller configured for modifying the lifestyle regimen of the user in a manner that is predicted to minimize the at least one error signal. As another example, the lifestyle optimizer may comprise a feature extraction component and a lifestyle regression model (e.g., a deep neural network). The feature extraction component may be configured for extracting a single-dimensional vector of lifestyle features from each different variation of the combination of lifestyle variables sequentially administered to the user and a single-dimensional vector of qualitative indicator features from the detected physiological activity of the user. The lifestyle regression model may have a first input, a second input, and a third input wherein the lifestyle optimizer is configured for modifying the lifestyle regimen of the user by inputting each single-dimensional vector of lifestyle features into the first input of the lifestyle regression model, inputting each single-dimensional vector of qualitative indicator features into the second input of the lifestyle regression model, and inputting a single-dimensional vector of target qualitative indicator features into the third input of the lifestyle regression model, such that the lifestyle regression model outputs a single-dimensional vector of lifestyle features.

In an optional embodiment, the lifestyle optimizer is configured for determining that the optimized lifestyle regimen has become non-optimal for the user, modifying values of the combination of lifestyle variables of the lifestyle regimen again, such that the peripheral device sequentially administers other different variations of the combination of lifestyle variables respectively having other different sets of values to the user, deriving other sets of qualitative indicators of the lifestyle aspect of the user from the detected physiological activity of the user respectively in response to the other different variations of combination of lifestyle variables sequentially administered to the user, and reoptimizing the lifestyle regimen of the user based on the other different variations of the combination of lifestyle variables and the other derived sets of qualitative indicators.

In one specific example, the lifestyle optimizer is a sleep quality optimizer, the lifestyle regimen is a sleep quality regimen, the lifestyle variables are sleep quality variables (e.g., at least one of going to bed at a specified time, waking up at a specified time, not drinking alcohol or caffeine after a specified time, not eating food after a specified time, utilizing blue light blocking glasses and deep-wave sound machine, performing an exercise routine at a specified time, stop working at a specified time, reading a book or watching a movie, meditation or breathwork, setting bedroom at a certain temperature at bedtime), the qualitative indicators are sleep quality indicators (e.g., a percentage breakdown between Light Sleep, Deep Sleep, REM, and Awake of the user or an amount of Deep Sleep of the user), and the lifestyle aspect is sleep of the user. The non-invasive measurement device(s) may be configured for detecting the physiological activity of the user while the user is sleeping, and the peripheral device may be configured for sequentially administering the different variations of the combination of sleep quality variables to the user while the user is awake.

In another specific example, the lifestyle optimizer is a biological age optimizer, the lifestyle regimen is a biological age regimen, the lifestyle variables are biological age variables, the qualitative indicators are biological age indicators (e.g., epigenic data, such as a pattern of DNA methylation of a genome of the user, or brain data), and the lifestyle aspect is biological age of the user. In one embodiment, the qualitative age indicators may comprise brain data, in which case, the non-invasive measurement device may comprise a non-invasive brain interface assembly, the detected physiological activity of the user may comprise detected brain activity of the user, and each derived set of qualitative indicators may comprise a physiological brain state of the user comprising the brain data. In another embodiment, the biological age indicators may be biological age indicators of different organs of the user, in which case, the biological age of the user may comprise biological ages of different organs of the user.

In still another specific example, the lifestyle optimizer is a mental energy expenditure optimizer, the lifestyle regimen is an efficient mental energy regimen, the lifestyle variables are mental energy expenditure variables (e.g., at least one of avoiding a person or situation, playing music at a specified time, playing a movie at a specified time, and conducting a meditative session at a specified time), the qualitative indicators are mental energy expenditure indicators (e.g., emotional brain states of the user), and the lifestyle aspect of the user is mental energy expenditure of the user. The non-invasive measurement device(s) may be configured for detecting the physiological activity of the user while the user is awake, and the peripheral device may be configured for sequentially administering the different variations of the combination of mental energy expenditure variables to the user while the user is awake. In one optional embodiment, the mental energy expenditure optimizer is configured for prompting the user to manually enter a value of a mental energy expenditure variable currently performed by the user, such that at least one variation of the combination of mental energy expenditure variables has the manually entered value.

In accordance with another aspect of the present inventions, a method of optimizing a lifestyle regimen of a person containing a combination of lifestyle variables is provided.

The method comprises repeatedly modifying at least one value of the combination of lifestyle variables, thereby creating different variations of the combination of lifestyle variables respectively having different sets of values, and sequentially administering the different variations of the combination of lifestyle variables to the person. An optional method further comprises allowing the person to manually enter a value of a lifestyle variable, such that at least one variation of the combination of lifestyle variables has the manually entered value. The value of the lifestyle variable may be currently performed by the person, in which case, the method may further comprise prompting the person to manually enter the currently performed value of the lifestyle variable.

The method further comprises detecting physiological activity of the person in response to the administration of the combination of lifestyle variables to the person, and deriving sets of qualitative indicators of an aspect of a lifestyle of the person from the detected physiological activity of the person.

In one advantageous method, the detected physiological activity of the person comprises detected brain activity of the person, and each derived set of qualitative indicators comprises a brain state of the person (e.g., physiological brain state of the person and/or mental brain state of the person). The brain activity of the person may be, e.g., optically detected or magnetically detected. One method comprises detecting the brain activity of the person comprises detecting energy from a brain of the person, and identifying the brain activity in response to detecting the energy from the brain of the user. In another method, the detected physiological activity of the comprises detected peripheral physiological activity (e.g., at least one of heart rate, heart rate variability, respiratory rate, blood pressure, blood flow, skin conductivity, blood glucose, Cortisol, DHEA-s, Serotonin, GABA, Dopamine, Norepinephrine, Epinephrine, and Glutamate).

The method further comprises optimizing the lifestyle regimen of the person based on the different variations of the combination of lifestyle variables and the derived sets of qualitative indicators (e.g., using machine-learning). One method further comprises selecting one of the different variations of the combination of lifestyle variables for the optimized lifestyle regimen.

In one method, the lifestyle regimen of the person is modified, such that at least one of the derived sets of qualitative indicators substantially matches a target set of qualitative indicators. For example, modifying the lifestyle regimen of the person may comprise comparing each derived set of qualitative indicators and the target set of qualitative indicators, respectively generating at least one error signal, and modifying the lifestyle regimen of the person in a manner that is predicted to minimize the at least one error signal. As another example, modifying the lifestyle regimen of the person comprises extracting a single-dimensional vector of lifestyle features from each different variation of the combination of lifestyle variables sequentially administered to the user and a single-dimensional vector of qualitative indicator features from the detected physiological activity of the person, inputting each single-dimensional vector of lifestyle features into a first input of a lifestyle regression model (e.g., a deep neural network), inputting each single-dimensional vector of qualitative indicator features into a second input of the lifestyle regression model, and inputting a single-dimensional vector of target qualitative indicator features into a third input of the lifestyle regression model, such that the lifestyle regression model outputs a single-dimensional vector of lifestyle features.

An optional method further comprises determining that the optimized lifestyle regimen has become non-optimal for the person, repeatedly modifying at least one value of the combination of lifestyle variables, thereby creating additional different variations of the combination of lifestyle variables respectively having additional different sets of values, sequentially administering the additional different variations of the combination of lifestyle variables of the lifestyle regimen to the person, detecting physiological activity of the person in response to the administration of the additional different variations of the combination of lifestyle variables to the person, deriving other sets of qualitative indicators of the lifestyle aspect of the person from the detected physiological activity of the person respectively in response to the other different variations of the combination of lifestyle variables sequentially administered to the person, and reoptimizing the lifestyle regimen of the person based on the other different variations of the combination of lifestyle variables and the other derived sets of qualitative indicators.

In one specific example, the lifestyle regimen is a sleep quality regimen, the lifestyle variables are sleep quality variables (e.g., at least one of going to bed at a specified time, waking up at a specified time, not drinking alcohol or caffeine after a specified time, not eating food after a specified time, utilizing blue light blocking glasses and deep-wave sound machine, performing an exercise routine at a specified time, stop working at a specified time, reading a book or watching a movie, meditation or breathwork, setting bedroom at a certain temperature at bedtime), the qualitative indicators are sleep quality indicators (e.g., a percentage breakdown between Light Sleep, Deep Sleep, REM, and Awake of the person or an amount of Deep Sleep of the person), and the lifestyle aspect is sleep quality of the person. The physiological activity of the person may be detected while the person is asleep, and the different variations of the combination of sleep quality variables may be sequentially administered to the person while the person is awake.

In another specific example, the lifestyle regimen is a biological age regimen, the lifestyle variables are biological age variables, the qualitative indicators are biological age indicators (e.g., epigenic data, such as a pattern of DNA methylation of a genome of the user, or brain data), and the lifestyle aspect is biological age of the user. In one method, the qualitative age indicators may comprise brain data. In another method, the biological age indicators may be biological age indicators of different organs of the user, in which case, the biological age of the user may comprise biological ages of different organs of the user.

In another specific example, the lifestyle regimen is an efficient mental energy regimen, the lifestyle variables are mental energy expenditure variables (e.g., at least one of avoiding a person or situation, playing music at a specified time, playing a movie at a specified time, and conducting a meditative session at a specified time), the qualitative indicators are mental energy expenditure indicators (e.g., emotional brain states of the person), and the lifestyle aspect of the person is mental energy expenditure of the person. The physiological activity of the person may be detected while the person is awake, and the different variations of the combination of mental energy expenditure variables may be sequentially administered to the person while the person is awake. One optional method further comprises prompting the person to manually enter a value of a mental energy expenditure variable currently performed by the person, such that at least one variation of the combination of mental energy expenditure variables has the manually entered value.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of a non-invasive self-autonomous system constructed in accordance with one embodiment of the present inventions;

FIG. 2 is a block diagram of one specific implementation of a lifestyle optimizer used in the non-invasive self-autonomous system of FIG. 1;

FIG. 3 is a block diagram of another specific implementation of a lifestyle optimizer used in the non-invasive self-autonomous system of FIG. 1;

FIG. 4 is block diagram of exemplary hardware used in a lifestyle optimizer used in the non-invasive self-autonomous system of FIG. 1;

FIG. 5 is a view of one specific physical embodiment of the non-invasive self-autonomous system of FIG. 1;

FIG. 6 is a view of another specific physical embodiment of the non-invasive self-autonomous system of FIG. 1;

FIGS. 7A-7D are views of exemplary non-invasive wearable devices as used with the non-invasive self-autonomous system of FIG. 6;

FIG. 8 is a view of still another specific physical embodiment of the non-invasive self-autonomous system of FIG. 1;

FIGS. 9A-9C are views of exemplary non-invasive wearable devices as used with the non-invasive self-autonomous system of FIG. 8; and

FIG. 10 is a flow diagram illustrating one method of optimizing a lifestyle regimen of a person in accordance with the present inventions.

### DETAILED DESCRIPTION OF THE EMBODIMENTS

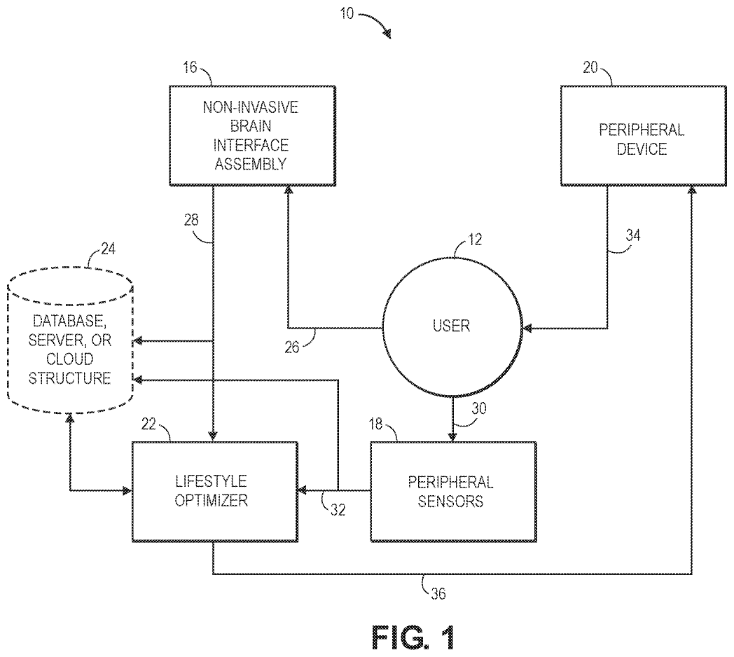

Referring now to FIG. 1, a generalized embodiment of a non-invasive self-autonomous system 10 constructed in accordance with the present inventions will be described. The non-invasive self-autonomous system 10 automatically generates and continually optimizes an Autonomous Self of a user 12 in a normal life and work environment, so that the user 12 may become the best version of themself. For the purposes of this specification, a “normal life and work environment” is an environment that is usual and ordinary, and thus, necessitates that the user 12 be able to freely ambulate without any physical hindrance by the system 10 or other system to which the system 10 is coupled or otherwise is an adjunct. Thus, a normal life and work environment excludes a clinical setting (e.g., any setting in which a conventional magnetic resonance imaging (MRI) machine or computed tomography (CT) could potentially be used to detect neural activity from the user 10). In alternative embodiments, the non-invasive self-autonomous system 10 may be non-portable and/or non-wearable in cases where it is suitable for the non-invasive self-autonomous system 10 to be operated outside of a normal life and working environment, e.g., in a research facility or laboratory.

The non-invasive self-autonomous system 10 generally comprises at least one non-invasive measurement device, and in the illustrated embodiment, a non-invasive brain interface assembly 16 and one or more peripheral sensors 18. The non-invasive measurement devices 16, 18 are configured for detecting physiological activity of the user 12.

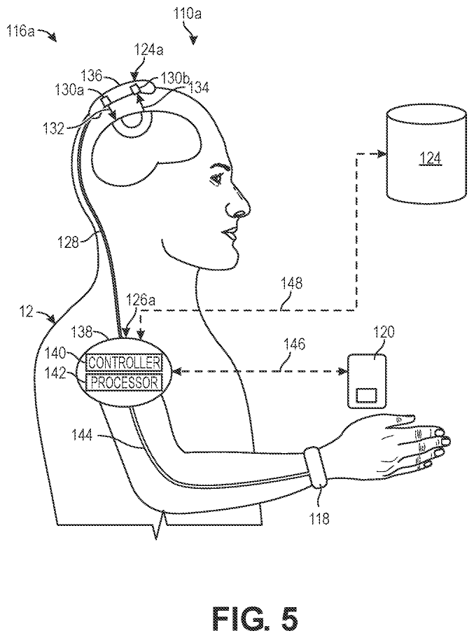

In particular, the non-invasive brain interface assembly 16 is configured for detecting physiological activity in the form of neural activity 26 in the brain 14 (shown in FIGS. 5, 6, and 8) of the user 12 (i.e., brain activity), and outputting physiological signals 28 characterizing the brain activity 26 of the user 12. The non-invasive brain interface assembly 16 may be any device capable of non-invasively acquiring hi-fidelity physiological signals 28 representing the brain activity 26 of the user 12. For example, as will be described in further detail below, the non-invasive brain interface assembly 16 may be optically-based, magnetically-based, multi-modal (e.g., both optical-based and electrical-based), or be based on any other modality that enables it to non-invasively detect brain activity 26 of the user 12 (i.e., through the intact skin and skull of the user 12), through the use of sensitive electronics. In the preferred embodiment, the non-invasive brain interface assembly 16 is portable and wearable, such that the user 12 may operate the non-invasive brain interface assembly 16 in a normal life and working environment.

The peripheral sensor(s) 18 are configured for detecting the physiological activity in the form of peripheral physiological activity 30 (i.e., physiological activity outside of the brain 14 of the user 12), and outputting physiological signals 32 characterizing the peripheral physiological activity 30 of the user 12 (e.g., heart rate, heart rate variability, respiratory rate, blood pressure, blood flow, skin conductivity, blood glucose, and/or Cortisol, DHEA-s, Serotonin, GABA, Dopamine, Norepinephrine, Epinephrine, and Glutamate levels, and genetics metabolomics and proteomics). In the illustrated embodiment, the peripheral sensor(s) 18 take the form of an optical wearable device, e.g., a wrist-worn device, such as, a Whoop strap, Fitbit, Garmin, Apple Watch, a time domain-based optical measurement system configured to non-invasively measure blood oxygen saturation (SaO2) through Time-Resolved Pulse Oximetry (TR-SpO2) (as described in U.S. Provisional Application Nos. 63/134,479, filed Jan. 6, 2021; 63/154,115, filed Feb. 26, 2021; 63/160,995, filed Mar. 15, 2021; 63/179,080, filed Apr. 23, 2021; and U.S. patent application Ser. No. 17/550,387, filed Dec. 14, 2021, which are all expressly incorporated herein by reference), Vo2 max, which is the maximum rate of oxygen consumption measured during incremental exercise; that is, exercise of increasing intensity (see e.g., https://www.healthline.com/health/vo2-maax), etc. Other types of optical wearable devices may include, a chest strap, an armband wearable device, a ring wearable on a finger, etc., that are configured for tracking a user's exercise performance and sleep patterns). Alternatively or additionally, the peripheral sensor(s) 18 may take the form of eye and facial trackers (for sensing facial expressions, such as blushing, frowning, smiling, yawning, grimacing, etc.), blood glucose monitors, Cortisol, DHEA-s, Serotonin, GABA, Dopamine, Norepinephrine, Epinephrine, Glutamate and PE monitors, voice recognition systems, keystroke capturing devices, etc. The peripheral sensor(s) 18 may also take the form of environment sensors for detecting ambient settings, e.g., temperature of the room or car, noise levels, etc. Alternatively or additionally, the peripheral sensor(s) 18 may take the form of separate imaging modalities such as ultrasound, skin scanning devices, or other forms of portable imaging modalities used for diagnostics purposes and gathering diagnostics data related to a person's health. Such diagnostic data may be stored in the database, server, or cloud structure 24 for access by the lifestyle optimizer 22. The diagnostic data may also come from the user's MRI results or other non-portable imaging platform and may be stored in the database, server, or cloud structure 24 for access by the lifestyle optimizer 22.

The non-invasive self-autonomous system 10 further comprises a peripheral device 20 (e.g., a Smartphone, tablet computer, or the like) configured for automatically administering a lifestyle regimen 34 containing a combination of lifestyle variables 34 to the user 12, e.g., on a daily basis. For example, the peripheral device 20 may actively provide the lifestyle regimen 34 in the form of different visual, auditory, or haptic stimuli to the user 12 (e.g., playing deep-wave sound, playing music, playing a movie or show, conducting a meditative session, etc.) and/or instruct the user 12 to perform the lifestyle regimen 34 (e.g., instructing the user 12 to avoid a certain person or situation, wear blue light blocking glasses, perform an exercise routine, stop working, read a book or watch a movie or show, perform meditation or breathwork, set bedroom environment at a certain temperature, go to bed at a specified time, wake up at a specified time, not drink alcohol after a specified time, not eating food after a specified time, etc.).

The peripheral device 20 can administer different product formulations to the user 12 in accordance with U.S. patent application Ser. No. 16/853,614, entitled “Non-Invasive System and Method for Product Formulation Assessment Based on Product-Elicited Brain State Measurements” (now U.S. Pat. No. 11,172,869), which is expressly incorporated herein by reference. One type of supplement for product formulation can be Nicotinamide mononucleotide (NMN) for anti-aging (see Tamas Kiss, et al., “Nicotinamide mononucleotide (NMN) Supplementation Promotes Anti-Aging miRNA Expression Profile in the Aorta of Aged Mice, Predicting Epigenetic Rejuvenation and Anti-Atherogenic Effects,” GeroScience, 2019 August; 41(4): 419-439 (https://www.ncbi.nlm.nih.gov/pmc/articies/PMC6815288).

The value of any particular lifestyle variable may be binary (i.e., either 0 (does not exist) or 1 (exists)). For example, a lifestyle regimen may play music (value=1) or may not play music at all (value=0). The value of at least one component of the lifestyle regimen may also be discrete or continuous. For example, a lifestyle regimen may play music at a selected time (e.g., 4 pm, 5 pm, 6 pm, etc. or at any time between the top of the hour).

The non-invasive self-autonomous system 10 further comprises a lifestyle optimizer 22 configured for instructing the peripheral device 20 to perform a series of experiments on the user 12, and optimizing a lifestyle regimen 34 of the user 12 based on these series of experiments. The lifestyle optimizer 22 may instruct the peripheral device 20 to administer any combination of different lifestyle variables in the form of a hypothesis for each experiment performed on the user 12.

Significantly, utilizing a suitable machine learning algorithm (e.g., a machine learning algorithm that provides a regression output and contains various components and layers that can include but are not limited to: classical machine learning models such as support vector machines, random forests, or logistic regression, as well as modern deep learning models such as deep convolutional neural networks, attention-based networks, recurrent neural networks, or fully connected neural networks), the lifestyle optimizer 22 may, in response to each experiment performed on the user 12, analyze the resulting physiological activity acquired from the user 12 (e.g., the physiological signals 28 characterizing the brain activity 26 of the user 12 output by the non-invasive brain interface assembly 16 and/or the physiological signals 32 characterizing the peripheral physiological activity 30 of the user 12 output by the peripheral sensor(s) 18), and generate these hypotheses (in the form of different combinations of lifestyle variables) that can then be used in subsequent experiments to optimize the lifestyle regimen 34 of the user 12 (i.e., by determining the values of the lifestyle variables that maximize the quality of at least one aspect of the lifestyle of the user 12). The combination of lifestyle variables with associated values that maximizes the quality of the aspect(s) of the lifestyle of the user 12 will be deemed the optimized lifestyle regimen for the user 12.

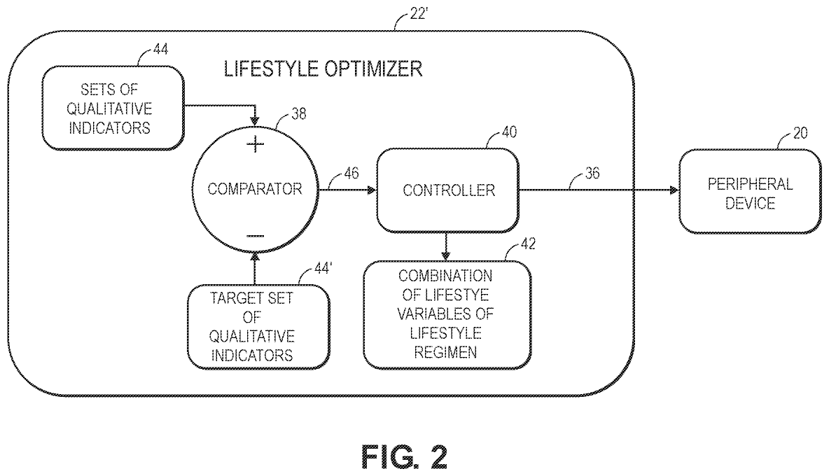

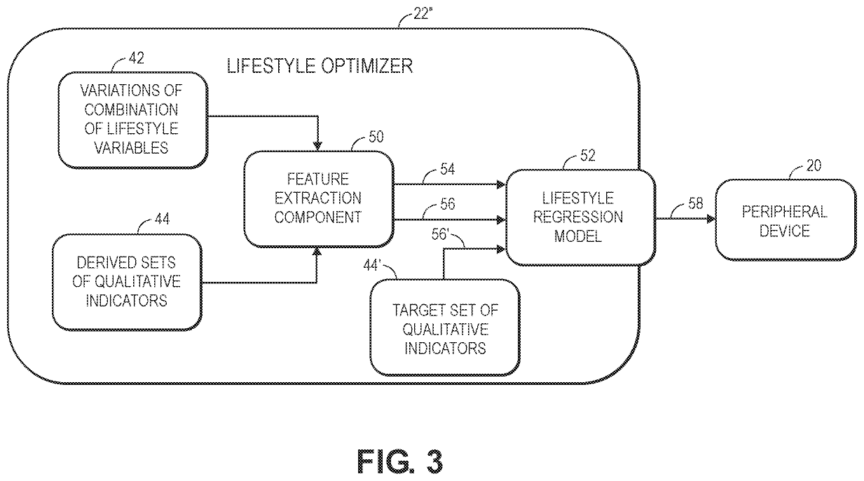

The lifestyle optimizer 22 is configured for optimizing the lifestyle regimen 34 of the user 12 by first modifying values of the combination of lifestyle variables of the lifestyle regimen 24 to create different variations of the combination of lifestyle variables 42 (shown in FIGS. 2 and 3) with different sets of values, and generating and outputting control signals 36 to the peripheral device 20 that instruct the peripheral device 20 to administer the different variations of the combination of lifestyle variables 42 of the lifestyle regimen 34 to the user 12. For example, the lifestyle optimizer 22 may change the time of a last meal of the user 12 from 6 pm to 5 pm, or may change the bedtime of the user 12 from 9:00 pm to 8:30 pm, or may change the music before bedtime of the user from jazz to soft rock, or may decide to play deep-wave sound when the user 12 is asleep, etc.

In an optional embodiment, the user 12 may manually enter values of lifestyle variables into the lifestyle optimizer 22 via the peripheral device 20, in which case, at least one variation of the combination of lifestyle variables 42 will include the manually entered values of the lifestyle variables, such that the lifestyle optimizer 22 may take these manually entered values of the lifestyle variables into account when subsequently optimizing the lifestyle regimen of the user 12. For example, the user 12 may perform a value of a lifestyle variable in contradiction to a lifestyle variable instructed by the peripheral device 20 to be performed by the user 12 (e.g., the peripheral device 20 may suggest to the user 12 to not consume coffee after 6 pm or to meditate, but the user 12 may have consumed coffee at 8 pm and did not meditate), in which case, the user 12 may enter the correct value of the lifestyle variable into the lifestyle optimizer 22 (e.g., the user 12 had actually consumed coffee at 8 pm, and had not meditated). Thus, the value of the lifestyle variable related to the consumption of coffee may be corrected by the user 12, such that the lifestyle optimizer 22 may subsequently optimize the lifestyle regimen 34 of the user 12 based on the previous occurrence of correct values for the lifestyle variables of the lifestyle regimen 34. As another example, the user 12 may enter additional lifestyle variables not addressed by the lifestyle regimen 34 generated by the lifestyle optimizer 22 (e.g., the user 12 exercises at 12 pm and had watched a movie when the lifestyle regimen 34 generated by the lifestyle optimizer 22 does not address exercise or watching moves at all). In another optional embodiment, the lifestyle optimizer 22 may instruct the peripheral device 20 to prompt the user 12 to enter the nature of an activity that the user 12 is currently performing (e.g., interacting with social media), such that at least one of the different variations of the combination of lifestyle variables 42 (corresponding to the nature of the activity currently performed by the user 12) includes the manually entered values of the lifestyle variables. Thus, the lifestyle optimizer 22 may subsequently optimize the lifestyle regimen 34 of the user 12 based on the previous occurrence of all available variables that can be used in the lifestyle regimen 34, including those not in the lifestyle regimen 34 previously generated by the lifestyle optimizer 22.

In response to the administration of the lifestyle regimen 34 administered to the user 12 by the peripheral device 20, the lifestyle optimizer 22 is further configured for deriving sets of qualitative indicators 44 (shown in FIGS. 2 and 3) of an aspect of a lifestyle of the user 12 from the detected physiological activity of the user 12 (e.g., the physiological signals 28 characterizing the brain activity 26 of the user 12 output by the non-invasive brain interface assembly 16 and/or the physiological signals 32 characterizing the peripheral physiological activity 30 of the user 12 output by the peripheral sensor(s) 18) respectively in response to the different variations of the combination of lifestyle variables 42 of the lifestyle regimen 34 administered to the user 12 by the peripheral device 20. Each set of qualitative indicators 44 may have only one qualitative indicator or multiple qualitative indicators 44.

One type of qualitative indicator is a brain state of the user 12, which may be derived from the physiological signals 28 characterizing the brain activity 26 of the user 12 output by the non-invasive brain interface assembly 16. The brain state of the user may be a physiological brain state (or low-level brain state) or a mental brain state (or high-level brain state). The physiological brain state may be a state of physiological activity in the brain 14 of the user 12, while the mental brain state may be an interpretation made by the brain in response to physiological activity in the brain 14 of the user 12.

A physiological brain state of the user 12 is defined by characteristics of the spatiotemporal brain activity that is captured, and can include, e.g., location or spatial pattern of neural activity, fine grained pattern within or across locations, amplitude of signal, timing of response to behavior, magnitude of frequency bands (Gamma, Beta, Alpha, Theta, and Delta) of the signal (taking the Fourier transform of the time series), ratio of magnitude of frequency bands, cross-correlation between time series of signal between two or more locations captured simultaneously, spectral coherence between two or more locations captured simultaneously, components that maximize variance, components that maximize non-gaussian similarity, etc. The characteristics of the brain activity can be extracted from preprocessed raw data, which typically involves filtering the raw detected data (either in the time domain or the frequency domain) to smooth, remove noise, and separate different components of signal.

In contrast, a mental brain state of the user 12 may include, e.g., an emotional state (e.g., joy, excitement, relaxation, surprise, anxiety, sadness, anger, disgust, contempt, fear, etc.), a cognitive state encompassing intellectual functions and processes (e.g., memory retrieval, focus, attention, creativity, reasoning, problem solving, decision making, comprehension and production of language, etc.), or a perceptive state (e.g., face perception, color perception, sound perception, visual perception, texture perception by touch etc.). The physiological signals 32 characterizing the peripheral physiological activity 30 of the user 12 output by the peripheral sensor(s) 18 may be used by the lifestyle optimizer 22 to inform the mental brain states determined by the lifestyle optimizer 22.

The lifestyle optimizer 22 may determine a mental brain state of the user 12 based on the detected brain activity (i.e., based on the physiological brain state in this case) in any one of a variety of manners.

For example, the lifestyle optimizer 22 may perform a univariate approach in determining the mental brain state of the user 12, i.e., the brain activity can be detected in a plurality (e.g., thousands) of separable cortical modules of the user 12, and the brain activity obtained from each cortical module can be analyzed separately and independently. As another example, the lifestyle optimizer 22 may perform a multivariate approach in determining the mental brain state of the user 12, i.e., the brain activity can be detected in a plurality (e.g., thousands) of separable cortical modules of the user 12, and the full spatial pattern of the brain activity obtained from the cortical modules can be assessed together.

The lifestyle optimizer 22 may use any one of a variety of models to classify the mental brain state of the user 12, which will highly depend on the characteristics of brain activity that are input onto the models. Selection of the characteristics of brain activity to be input into the models must be considered in reference to univariate and multivariate approaches, since the univariate approach, e.g., focuses on a single location, and therefore will not take advantage of features that correlate multiple locations. Selecting a model will be heavily dependent on whether the data is labeled or unlabeled (meaning is it known what the user 12 is doing at the time that the brain activity is detected), as well as many other factors (e.g., is the data assumed to be normally distributed, is the data assumed relationship linear, is the data assumed relationship non-linear, etc.) Models can include, e.g., support vector machines, expectation maximization techniques, naïve-Bayesian techniques, neural networks, simple statistics (e.g., correlations), deep learning models, pattern classifiers, etc.

These models are typically initialized with some training data (meaning that a calibration routine can be performed on the user 12 to determine what the user 12 is doing). If no training information can be acquired, such models can be heuristically initialized based on prior knowledge, and the models can be iteratively optimized with the expectation that optimization will settle to some optimal maximum or minimum solution. Once it is known what the user 12 is doing, the proper characteristics of the neural activity and proper models can be queried. The models may be layered or staged, so that, e.g., a first model focuses on pre-processing data (e.g., filtering), the next model focuses on clustering the pre-processed data to separate certain features that may be recognized to correlate with a known activity performed by the user 12, and then the next model can query a separate model to determine the mental brain state based on that user activity.

The training data or prior knowledge of the user may be obtained by providing known life/work context to the user. Altogether, the models can be used to track mental state and perception under natural or quasi-natural (i.e., in response to providing known life/work context to the user) and dynamic conditions taking in the time-course of averaged activity and determining the mental state of the user based on constant or spontaneous fluctuations in the characteristics of the brain activity extracted from the data.

A set of data models that have already been proven, for example in a laboratory setting, can be initially uploaded to the non-invasive self-autonomous system 10, which the lifestyle optimizer 22 will then use to determine the mental brain state of the user 12. Optionally, the non-invasive self-autonomous system 10 may collect data during actual use with the user 12, which can then be downloaded and analyzed in a separate server, for example in a laboratory setting, to create new or updated models. Software upgrades, which may include the new or updated models, can be uploaded to the non-invasive self-autonomous system 10 to provide new or updated data modelling and data collection.

Further details regarding determining the mental brain state of a person based on detected brain activity can be found in a variety of peer-reviewed publications. See, e.g., Lee, B. T., Seok, J. H., Lee., B. C, Cho, S. W., Chai, J. H., Choi, I. G., Ham, B. J., “Neural correlates of affective processing in response to sad and angry facial stimuli in patients with major depressive disorder,” Prog Neuropsychopharmacol Biol Psychiatry, 32(3), 778-85 (2008); A. C. Felix-Ortiz, A. C., Burgos-Robles, A., Bhagat, N. D., Leppla, C. A., Tye, K. M., “Bidirectional modulation of anxiety-related and social behaviors by amygdala projections to the medial prefrontal cortex,” Neuroscience 321, 197-209 (2016); Beauregard, M., Levesque, J. & Bourgouin, P., “Neural correlates of conscious self-regulation of emotion,” J. Neurosci. (2001): 21, RC165; Phan, K. L., Wager, T., Taylor, S. F. & Liberzon, I., “Functional neuroanatomy of emotion: a meta-analysis of emotion activation studies in PET and fMRI,” Neuroimage, 16, 331-348 (2002); Canli, T. & Amin, Z., “Neuroimaging of emotion and personality: scientific evidence and ethical considerations,” Brain Cogn., 50, 414-431 (2002), McCloskey, M. S., Phan, K. L. & Coccaro, E. F., “Neuroimaging and personality disorders,” Curr. Psychiatry Rep., 7, 65-72 (2005); Heekeren, H. R., Marrett, S., Bandettini, P. A. & Ungerleider, L. G., “A general mechanism for perceptual decision-making in the human brain,” Nature, 431, 859-862 (2004); Shin L M, Rauch S L, Pitman R K. Amygdala, Medial Prefrontal Cortex, and Hippocampal Function in PTSD, Ann N Y Acad Sci., 1071(1) (2006); Lis E, Greenfield B, Henry M, Guile J M, Dougherty G., “Neuroimaging and genetics of borderline personality disorder: a review,” J Psychiatry Neurosci., 32(3), 162-173 (2007); Etkin A, Wager T D, “Functional neuroimaging of anxiety: a meta-analysis of emotional processing in PTSD, social anxiety disorder, and specific phobia,” Am J Psychiatry, 164(10), 1476-1488 (2007); Etkin A. Functional Neuroimaging of Major Depressive Disorder: A Meta-Analysis and New Integration of Baseline Activation and Neural Response Data, Am J Psychiatry, 169(7), 693-703 (2012); Sheline Y I, Price J L, Yan Z, Mintun M A, “Resting-state functional MRI in depression unmasks increased connectivity between networks via the dorsal nexus, Proc Natl Acad Sci., 107(24), 11020-11025 (2010); Bari A, Robbins T W, “Inhibition and impulsivity: Behavioral and neural basis of response control,” Prog Neurobiol., 108:44-79 (2013); Kagias, Konstantinos et al. “Neuronal responses to physiological stress,” Frontiers in genetics, 3:222 (2012).

As will be described in further detail below, other types of qualitative indicators 44, besides a brain state, may be derived from the physiological signals 28 characterizing the brain activity 26 of the user 12 output by the non-invasive brain interface assembly 16, the brain state, itself, or the peripheral physiological activity 30 from the physiological signals 32 characterizing the peripheral physiological activity 30 of the user 12 output by the peripheral sensor(s) 18.

The lifestyle optimizer 22 is further configured for optimizing the lifestyle regimen 34 of the user 12 based on the different variations of the combination of lifestyle variables 42 and the derived sets of qualitative indicators 44 of the lifestyle aspect of the user 12. For example, if the derived sets of qualitative indicators 44 are consistently different from a target set of qualitative indicators 44, the lifestyle optimizer 22 may select different sets of values for the combination of lifestyle variables of the lifestyle regimen 34 to create the different variations of the combination of the lifestyle variables 42 that will be administered to the user 12 to evoke different sets of qualitative indicators 44 that are more consistent with the target set of qualitative indicators 44. Or, if the derived sets of qualitative indicators 44 are consistently the same as each other, the lifestyle optimizer 22 may select different sets of values for the combination of lifestyle variables of the lifestyle regimen 34 to create the different variations of the combination of the lifestyle variables 42 that will be administered to the user 12 in order to evoke sets of qualitative indicators 44 that are more varied relative to each other. In one embodiment, the lifestyle optimizer 22 may be configured for selecting one of the different sets of values (i.e., the set of values corresponding to the set of qualitative indicators 44 that best matches the target set of qualitative indicators 44) for the combination of lifestyle variables of the optimized lifestyle regimen 34. In another embodiment, the lifestyle optimizer 22 may be configured for selecting a set of values different from the different sets of values (e.g., by interpolating between two sets of values corresponding to the sets of qualitative indicators 44 that straddle the target set of qualitative indicators 44) for the combination of lifestyle variables of the optimized lifestyle regimen 34.

Once the lifestyle regimen 34 of the user 12 is optimized, the lifestyle optimizer 22 may instruct the peripheral device 20 to periodically (e.g., daily) administer the optimized lifestyle regimen 34 to the user 12. In one embodiment, after the lifestyle regimen 34 of the user 12 has been optimized, the lifestyle optimizer 22 may periodically monitor the lifestyle aspect of the user 12, and if it is determined that the lifestyle aspect of the user 12 has degraded, the lifestyle optimizer 22 may perform additional experiments on the user 12 to reoptimize the lifestyle regimen 34 of the user 12.

In particular, the lifestyle optimizer 22 may be configured for monitoring the sets of qualitative indicators 44 of the lifestyle aspect of the user 12 derived from the detected physiological activity (e.g., the physiological signals 28 characterizing the brain activity 26 of the user 12 output by the non-invasive brain interface assembly 16 and/or the physiological signals 32 characterizing the peripheral physiological activity 30 of the user 12 output by the peripheral sensor(s) 18), determining that the optimized lifestyle regimen 34 becomes non-optimal for the user 12, modifying values of the combination of lifestyle variables of the lifestyle regimen 34 again, such that the peripheral device 20 administers other variations of the combination of lifestyle variables 42 with other different sets of values 42 to the user, deriving other sets of qualitative indicators 44 of the lifestyle aspect of the user 12 from the detected physiological activity of the user 12 respectively in response to the other different variations of the combination of lifestyle variables 42 administered to the user 12, and reoptimizing the lifestyle regimen 34 of the user 12 based on the different sets of values 42 of the other variations of the combination of lifestyle variables 42 and the other derived sets of qualitative indicators 44 of the lifestyle aspect 24 of the user 12.

The lifestyle optimizer 22 may be configured for the lifestyle regimen 34 of the user 12 using machine-learning either on-line, meaning that the lifestyle regimen 34 of the user 12 is serially and continually updated or modified as each variation of the combination of lifestyle variables 42 and each derived set of qualitative indicators 44 of the lifestyle aspect 24 of the user 12 become available; or off-line, meaning that many different variations of the combination of lifestyle variables 42 and derived sets of qualitative indicators 44 of the lifestyle aspect 24 of the user 12 are accumulated or batched over a period of time (e.g., over a week), and then concurrently used to optimize the lifestyle regimen 34 of the user 12.

The advantage of using an on-line machine learning technique to optimize (or reoptimize) the lifestyle regimen 34 of the user 12 is that it can be used when it is computationally infeasible to optimize the lifestyle regimen 34 of the user 12 over a relative large amount of data (i.e., many different variations of the combination of lifestyle variables 42 and many derived sets of qualitative indicators 44), and furthermore, can dynamically adapt to new patterns in the different variations of the combination of lifestyle variables 42 and many derived sets of qualitative indicators 44 or many variations of the combination of lifestyle variables 42 and many derived sets of qualitative indicators 44 that change as a function of time. In contrast, the advantage of using an off-line machine learning technique to optimize the lifestyle regimen 34 of the user 12 is that the lifestyle regimen 34 of the user 12 may be optimized in a more robust manner, such that the lifestyle regimen 34 of the user 12 may be better optimized.

The non-invasive self-autonomous system 10 may optionally comprise a database, server, or cloud structure 24 configured for tracking the brain activity 26 and peripheral physiological activity 30 of the user 12. For example, the database, server, or cloud structure 24 may be configured to collect raw data (e.g., brain activity and peripheral physiological data) detected by the non-invasive brain interface assembly 16 and peripheral sensor(s) 18. Furthermore, the database, server, or cloud structure 24 (independently of or in conjunction with the functions of the lifestyle optimizer 22) may be configured for performing a data analysis of the raw data in order to determine the mental brain state of the user 12.

For example, if the raw data obtained by the user 12 is being anonymized and stored in the database, server, or cloud structure 24, the data models can be pooled across various users, which deep learning algorithms would benefit from. The database, server, or cloud structure 24 may be configured for performing cross-correlation analysis of the signal data analysis in order to reduce the pool size of the database and focus subject averaged data to a pool that is similar to the user. Most likely, each user will have a portion of their model optimized to them, but then another portion takes advantage of patterns extracted from a larger pool of users. It should also be appreciated that each user may perform any variety of an infinite number of activities. Thus, even if a user is properly calibrated, such calibration will only be for a small set of infinite possibilities. Generalizing models may comprise various variabilities and optimizing may be difficult. However, by building a large user database on the database, server, or cloud structure 24, a data analysis pipeline connected to such database, server, or cloud structure 24 can preprocess data (clean it up), extract all different kinds of features, and then apply an appropriate data model, to overcome this issue. The brain activity 26 and peripheral physiological activity 30 of the user 12 may be tracked with additional life/work context provided by the peripheral device 20 to acquire meta data in depth assessment of awareness and behavior modulation patterns of the user 12. Although, all of the tracked data analysis has been described as being performed by the database, server, or cloud structure 24, it should be appreciated that at least a portion of the tracked data analysis functionality may be locally incorporated elsewhere in the non-invasive self-autonomous system 10, with the caveat that it is preferred that the tracking of the brain activity and peripheral physiological activity between a pool of users be performed by the database, server, or cloud structure 24.

Referring now to FIG. 2, one embodiment of a lifestyle optimizer 22′ that can be used in the non-invasive self-autonomous system 10 employs an on-line machine learning technique to optimize the lifestyle regimen 34 of the user 12. To this end, the lifestyle optimizer 22′ comprises a comparator 38 configured for sequentially comparing the derived sets of qualitative indicators 44 of the lifestyle aspect of the user 12 and a target set of qualitative indicators 44′ for the lifestyle aspect of the user 12, and outputting error signals 46. The target sets of qualitative indicators 44′ is deemed to be the set of qualitative indicators of an optimized aspect of the lifestyle of the user 12. In the illustrated embodiment, the derived sets of qualitative indicators 44 and target sets of qualitative indicators 44′ are quantified, such that the error signals 46 sequentially output by the comparator 38 are representative of the respective differences between the derived sets of qualitative indicators 44 and a target set of qualitative indicators 44′. For example, features can be extracted from the derived sets of qualitative indicators 44 and a target set of qualitative indicators 44′ and arranged in single-dimensional vectors.

The lifestyle optimizer 22′ further comprises a controller 40 configured for modifying the values of the lifestyle variables of the lifestyle regimen 34 of the user 12 in a manner that is predicted to minimize the error signals 46. In the illustrated embodiment, the controller 40 is configured for sequentially (e.g., daily) instructing the peripheral device 20 to administer the lifestyle regimen 34 to the user (i.e., selecting different sets of values for the combination of lifestyle variables of the lifestyle regimen 34 to create different variations of the combination of lifestyle variables 42 to be administered to the user 12 in order to evoke different physiological activity in the user 12). For each comparison between a derived set of qualitative indicators 44 and target set of qualitative indicators 44 made by the comparator 38 to yield an error signal 46, the controller 40 is further configured for selecting or modifying the values of the combination of lifestyle variables of lifestyle regimen 34, thereby creating the different variations of the combination of lifestyle variables 42, and generating control signals 46 that instruct the peripheral device 20 to administer the different variations of the combination of lifestyle variables 42 to the user 12 in a manner that is predicted to minimize the error signal 46, i.e., in a manner that a subsequently derived set of qualitative indicators 44 substantially matches (e.g., within a 10 percent error) the target set of qualitative indicators 44.

Referring now to FIG. 3, another embodiment of a lifestyle optimizer 22″ that can be used in the non-invasive self-autonomous system 10 employs an off-line machine learning technique to optimize the lifestyle regimen 34 of the user 12. Conceptually, the lifestyle optimizer 22″ can be considered to be a specific embodiment of the lifestyle optimizer 22′ illustrated in FIG. 2, the difference being that, instead of the qualitative indicator comparison and error generation functions previously performed by the lifestyle optimizer 22′, these functions are illustrated as being performed by a machine learning algorithm associated with a lifestyle regression model 52, such as, e.g., deep neural network.

A feature extraction component 50 is configured for extracting lifestyle features from the different variations of the combination of lifestyle variables 42 of each lifestyle regimen 34 administered by the peripheral device 20 to the user 12, and outputting single-dimensional vectors of lifestyle features 54, and extracting qualitative indicator features from the derived set of qualitative indicators 44, and outputting single-dimensional vectors of qualitative indicator features 56.

The lifestyle regression model 52 has a first input for receiving the single-dimensional vectors of lifestyle features 54 from the feature extraction component 50, a second input for receiving the single-dimensional vectors of qualitative indicator features 56 from the feature extraction component 50, a third input for receiving a single-dimensional vector of target qualitative indicator features 56′ characterizing the target set of qualitative indicator features 44′, and an output for sending a single-dimensional vector of optimized lifestyle features 58 characterizing the different variations of the lifestyle variables 42 of an optimized lifestyle regimen 34 to the peripheral device 20. The output for sending a single-dimensional vector of optimized lifestyle features 58 can be set as the control signal 36 illustrated in FIG. 1.

A machine learning algorithm can also include, e.g., Gradient Descent, where a group of a random subset of the whole training data (which consist of all of the inputs (e.g., the extracted lifestyle features, extracted qualitative indicator features, and target qualitative indicator features of the user 12) and all of the outputs (e.g., the lifestyle features of an optimized lifestyle regimen 34)) are used to adjust the parameters, then another random subset is used to adjust the parameters, until the difference is less and less. The processing of the data in this fashion takes place inside the model. Not all machine learning methods use Gradient Descent, but known machine leaning algorithms adjust parameters with an optimizer, e.g., stochastic gradient descent, Newtonian methods, matrix inversion (such as in least squares fitting). The model can include an optimization component and the optimization component takes the whole input/output data and an optimization algorithm and it optimizes the parameters of the model.

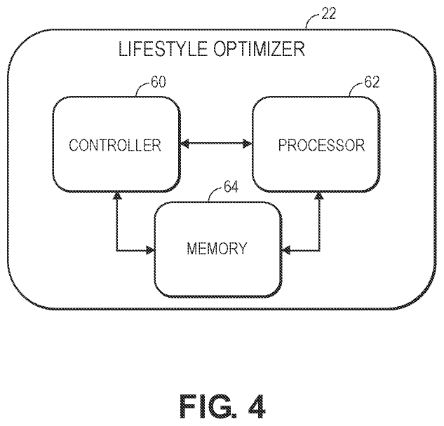

In one embodiment illustrated in FIG. 4, the lifestyle optimizer 22 may be any computing device, e.g., one that comprises a controller 60, a processor 62, a memory 64, a display (not shown), and an input device (not shown). The lifestyle optimizer 22 can, e.g., be a computer, tablet, mobile device, or any other suitable device for processing information. The lifestyle optimizer 22 can be local to the user 12 or can include components that are non-local to the user 12. For example, in at least some embodiments, the user 12 may operate a terminal that is connected to a non-local computing device. In other embodiments, the memory 64 can be non-local to the user 12. The lifestyle optimizer 22 can utilize any suitable processor 62, including one or more hardware processors that may be local to the user or non-local to the user or other components of the lifestyle optimizer 22. The processor 62 is configured to execute instructions provided to the processor 62, as described below.

Any suitable memory 64 can be used for the lifestyle optimizer 22. The memory 64 can be a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal. The term “modulated data signal” can include a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display can be any suitable display device, such as a monitor, screen, or the like, and can include a printer. In some embodiments, the display is optional. In some embodiments, the display may be integrated into a single unit with the lifestyle optimizer 22, such as a tablet, smart phone, or smart watch. The input device can be, for example, a keyboard, mouse, touch screen, track ball, joystick, voice recognition system, or any combination thereof, or the like.

Although the controller 60 and processor 62 are described herein as being separate components, it should be appreciated that portions or all functionality of the controller 60 and processor 62 may be performed by a single component. Furthermore, although all of the functionality of the controller 60 is described herein as being performed by a single component, and likewise all of the functionality of the processor 62 is described herein as being performed by a single component, such functionality each of the controller 60 and the processor 62 may be distributed amongst several components. It should also be appreciated that all or a portion of the controller 60 may be located outside of a physical computing device, e.g., as a Field Programmable Gate Array (FPGA). All or a portion of the controller 60 and the processor 62 may be located in another component of the non-invasive self-autonomous system 10. For example, some of the functionality of the lifestyle optimizer 22, e.g., the functionality of deriving the brain state of the user 12 may be alternatively located in the non-invasive brain interface assembly 16. It should be appreciated that those skilled in the art are familiar with the terms “controller” and “processor,” and that they may be implemented in software, firmware, hardware, or any suitable combination thereof.

The non-invasive self-autonomous system 10 may be modified to improve any other aspect, or all aspects, of the lifestyle of the user 12.

For example, in one embodiment, the non-invasive self-autonomous system 10 may be specially configured for improving the sleep quality of the user 12, e.g., to maximize the amount of Deep Sleep of the user 12. The non-invasive self-autonomous system 10 will determine a lifestyle regimen 34, and in this case a sleep quality regimen, that produces the perfect night of sleep for the user 10. The lifestyle optimizer 22 may take the form of a specialized sleep quality optimizer.

As provided in “Understanding Sleep Cycles and the States of Sleep,” (Nov. 1, 2019) (https://www.whoop.com/thelocker/stages-of-sleep-cycles), the average healthy adult will experience 3-5 sleep cycles per night, and within those cycles, there are four distinct sleep stages: Light Sleep, Deep/Slow Wave Sleep (SWS), Rapid Eye Movement (REM), and Awake. Light Sleep represents the physiological process taken to transition to SWS, also known as Deep Sleep. Deep Sleep is the time when muscles are repaired and grow. During this stage the body produces 95% of its daily supply of growth hormones. As stated by Mark Van Heusen, “Deep Sleep: What it is, Benefits of it, How Much Do you Need & How to Increase it,” (Dec. 1, 2019) (https://www.whoop.com/thelocker/what-is-deep-sleep), Deep Sleep is also the time when the immune system is strengthened, cells regenerate, tissue and bone are repaired, blood flow to muscles is increased, metabolism and blood sugar levels are balanced, and the brain is detoxified. REM Sleep is when the brain is restored, and is the time that ideas and skills acquired during the day are cemented as memories. Awake is included as a sleep stage, because it is natural to be awake for brief periods many times during the night. Each sleep cycle is entered through Light Sleep, transitions to SWS or Deep Sleep within about 10 minutes, and then to REM Sleep somewhere around 90 minutes after falling asleep. Awake will follow and a new sleep cycle will begin from there. The amount of time a person will spend in each sleep stage varies night by night. In general, a healthy breakdown to aim for is 50% Light Sleep, 23% SWS (Deep Sleep), 22% REM, and 5% Awake.

According to Mark Van Deusen, “Sleeping Tips From the 100 Best Sleepers on WHOOP” (Jan. 16, 2020) (https://www.whoop.com/thelocker/sleeping-tips-from-100-best-sleepers), there are many things that a person can do or refrain from doing to improve sleep. For example, a person may create an ideal sleep environment by having a quiet and dark room and weighted blanket, using an eye mask, ear plugs, and sleeping alone, using blue light blocking glasses, listening to floating and binaural beats, having a great mattress/pillow, using an extra pillow, turning on a fan in the background, having a very cold room with a nice blanket, and having an empty bladder. A person may also exercise better pre-bedtime activities including winding down to lower heart rate, taking a bath in the evening, taking a warm shower or using a heating pad before bed, relaxing at least an hour before bed, setting a reminder an hour before bedtime to prepare to sleep, working out hard, stretching right before bedtime, making a list of tasks to perform the next day, perform meditative breathing or meditation, refrain from looking at social media, reading before bed, turning off television, listening to relaxing music, making it a point to go to bed at the same time, etc. A person may also exercise better daytime habits including waking up naturally, consistent morning routine, physical exertion during the day, reducing stress and prioritizing recovery, waking up at least three hours before work, avoiding naps in afternoon, avoiding drinking alcohol, etc. A person may also exercise better supplement and nutritional habits including eating quality carbohydrates before bedtime, limiting liquid consumption before bedtime, using CBD+recovery balm and LUSH sleepy lotion, hydrating oneself, drinking chamomile or turmeric-based tea before bedtime, preparing meals for the next day, etc.

In the illustrated embodiment, the optimized sleep quality regimen 34 is considered by the sleep quality optimizer 22 to be the sleep quality regimen that results in the best night of sleep for the user 12, in which case, the lifestyle variables are sleep quality variables (e.g., going to bed at a specified time, waking up at a specified time, not drinking alcohol or caffeine after a specified time, not eating food after a specified time, performing an exercise routine at a specified time, stop working at a specified time, reading a book or watching a movie, performing meditation or breathwork, setting bedroom at a certain temperature at bedtime, etc.), the lifestyle aspect of the user 12 of which the set of qualitative indicators 44 is derived is sleep quality of the user 12, the derived set of qualitative indicators 44 may be sleep quality indicators (e.g., of a sleep cycle breakdown of the user 12), and the target set of qualitative indicators 44′ may be target sleep quality indicators (e.g., of a healthy sleep cycle breakdown of 50% Light Sleep, 23% SWS (Deep Sleep), 22% REM, and 5% Awake or simply an amount of Deep Sleep of the user 12). In the illustrated embodiment, the sleep quality regimen 34 is administered to the user 12 when the user 12 is awake, while the set of sleep quality indicators 44 are derived from physiological signals detected in the user 12 while the user 12 is asleep.

In one embodiment, the set of qualitative indicators 44 are derived from the physiological brain state of the user 12 acquired from the physiological signals 28 output by the non-invasive brain interface assembly 16, and in particular, the frequency bands (Gamma, Beta, Alpha, Theta, and Delta) of the brain of the user 12 during sleep, which is indicative of the sleep states of the user 12. As the user 12 enters Light Sleep, Alpha waves are replaced with slower Theta Waves. During SWS (Deep Sleep), brain activity further slows down as Delta waves occur. During REM, the pattern of brain waves is similar to those that occur during Light Sleep. During Awake, Beta waves dominate. The set of derived sleep quality indicators 44 can be informed by the peripheral physiological signals 32 output by the peripheral sensor(s) 18. For example, the peripheral sensor(s) 18 may take the form of an optical wearable device, e.g., a wrist-worn device, such as, a Whoop strap, which can measure the heart rate, respiratory rate, blood pressure, blood flow, and skin conductivity, which can be correlated to the sleep stages of the user 12 (see Emily Capodilupo, “How Does WHOOP Measure Sleep, and How Accurate is It?” (Feb. 20, 2020) (https://www.whoop.com/thelocker/how-well-whoop-measures-sleep). Thus, the sleep quality optimizer 22 may derive, from physiological signals 28 output by the non-invasive brain interface assembly 16 and the peripheral physiological signals 32 output by the peripheral sensor(s) 18, a set of sleep quality indicators 44 in the form of the existence and duration of each of the sleep stages in each cycle experienced by the user 12.

As another example, as a proxy of the quality of sleep of the user 12 during the previous night, the sleep quality optimizer 22 may determine the mental brain state of the user 12 based on cognitive tests administered to the user 12 during daytime when the user 12 is awake (see Bryan Johnson, “Sleep and Impulse Control” (https://kernel.com/news/sleep-and-impulse-control), and as described in U.S. Provisional Application Ser. No. 63/154,123, filed Feb. 26, 2021; and U.S. Provisional Application Ser. No. 63/179,957, filed Apr. 26, 2021. In one embodiment, the peripheral device 20 administers an inhibitory reflex test (e.g., an anti-saccade test or a go/no-go test) to the user 12 while the non-invasive brain interface assembly 16 detects a physiological brain state of the user 12, and in particular, detects brain activity in a frontal lobe of the brain of the user 12. The variability in neural activation in the brain of the user 12 during an inhibitory reflex test can be highly correlated to the duration of total sleep and duration of Deep Sleep that the user 12 had the previous night.

In a similar manner described above with respect to the generic lifestyle regimen 34 of the user, the sleep quality optimizer 22 is configured for modifying values of the combination of sleep quality variables of the sleep quality regimen 34, thereby creating different variations of the combination of sleep quality variables 42 with different sets of values, instructing the peripheral device 20 to administer the different variations of combination of lifestyle variables 42, deriving sets of sleep quality indicators 44 of the user 12 from the detected physiological activity of the user 12 respectively in response to the different variations of the combination of sleep quality variables 42 administered to the user 12, and optimizing the sleep quality regimen 34 of the user 12 based on the different variations of the combination of sleep quality variables 42 of the sleep quality regimen 34 and the derived sets of sleep quality indicators 44 of the user 12.

In another embodiment, rather than focusing on sleep quality of the user 12, the non-invasive self-autonomous system 10 may be specially configured for reducing the biological age or decreasing the rate of the biological aging of the user 12, e.g., to maximize the life span and quality of life for the user 12. The non-invasive self-autonomous system 10 will determine a lifestyle regimen 34, and in this case a biological age reducing regimen, that reduces the biological age and/or decreases the rate of the biological aging of the user 12. The lifestyle optimizer 22 may take the form of a specialized biological age optimizer. In the illustrated embodiment, the optimized biological age reducing regimen is considered by the biological age optimizer 22 to be the biological age reducing regimen that results in the lowest biological age or lowest rate of biological aging that the user 12 can achieve, in which case, the lifestyle variables are biological age variables (e.g., sleep quality (as discussed above), eating a certain amount of fruits and vegetables, not eating a certain amount of red meat or fatty foods, not drinking a certain amount of alcohol, running or bicycling for a certain duration, going to the gym a certain number of times a week, fasting a certain amount of times and a certain time of day, etc.), the lifestyle aspect of the user 12 of which the set of qualitative indicators 44 is derived is biological age of the user 12, the derived set of qualitative indicators 44 may be biological age indicators.

The biological age indicators (qualitative indicators 44), may be, e.g., epigenic data (i.e., data characterizing changes in genes caused by behaviors and environment). As described in http://goop.com/wellness/health/what-is-my-biological-age/, one type of epigenic data takes the form of DNA methylation, which is a chemical modification to DNA, which, although not changing the sequence of the DNA, such chemical modification regulates which genes get turned on and which genes get turned off. There exists specific patterns of DNA methylation across an entire genome, with specific areas of the genome where there is an increase in methylation with age, and other specific areas of the genome where there is a decrease in methylation with age. A particular pattern of DNA methylation can be analyzed to estimate the biological age of a person based on hundreds of thousands of sites in the genome that are a reflection of overall health and function of that person. An at-home test exists that conveniently analyzes the pattern of DNA methylation of genomes acquired from a saliva sample to measure the biological age of a person. Alternatively, although not as convenient, the biological age of a person can be determined by analyzing the pattern of DNA methylation of genomes acquired from a blood sample in a laboratory setting.

Significantly, by analyzing the pattern of DNA methylation of genomes acquired from different parts of a body, the biological ages of the different body parts of a particular person can be estimated, thereby allowing a more nuanced understanding of the biological age of a person across different organs to provide a more comprehensive understanding of the overall health and aging of the person. For example, a pattern of DNA methylation of genomes acquired from blood may yield a particular biological age for a person, while a pattern of DNA methylation of genomes acquired from skin sample, cheek sample, or saliva sample may yield different biological age of the same person. Although not as practical, biopsies can be taken from the liver, heart, and brain, e.g., and different biological ages for these organs can be estimated based on the analysis of the patterns of DNA methylation of genomes acquired from these biopsies.

Other types of epigenic data besides DNA methylation may include an accumulation of genetic damage, telomere length, and telomere attrition (see Franke, K., et al., “Ten Years of Brain AGE as a Nueuroimaging Biomarker of Brain Aging: What Insights Have We Gained?”, Frontiers in Neurology, August 2019, Vol. 10, Article 789).

Although not as convenient as epigenic data, alternative biological age indicators may include any combination of c-reactive protein (CRP), total cholesterol, albumin, creatine, hbalc (average blood sugar), alkaline phosphatase, and urea nitrogen, which all require a blood sample to be taken from a person (see https://goop.com/wellness/health/what-is-my-biological-age/).

In another embodiment, the biological age indicators may take the form of neuroimages, e.g., Magnetic Resonance Imaging (MRI) data sets, which can be analyzed to estimate the biological age of a brain of a person. Machine learning may be applied to high-dimensional MRI datasets to build predictive statistical models of brain ageing, which models assume a trajectory of brain ageing that represents an individual's accumulation of deleterious changes that lead to alterations in brain function and increased risk of cognitive decline and disease (see James H. Cole, et al, “Brain Age and Other Bodily ‘Ages’: Implications for Neuropsychiatry,” Molecular Psychiatry (2019) 24: 266-281; Franke, K., et al., “Ten Years of Brain AGE as a Nueuroimaging Biomarker of Brain Aging: What Insights Have We Gained?”, Frontiers in Neurology, August 2019, Vol. 10, Article 789). Alternatively, the non-invasive brain interface assembly 16 can be conveniently utilized to generate physiological signals 28 a physiological brain state of the user 12 in the form of datasets that can be analyzed using machine learning to estimate the biological age of the brain of the user 12.

Thus, in a similar manner described above with respect to the generic lifestyle regimen 34 of the user, the biological age optimizer 22 is configured for modifying values of the combination of sleep quality variables of the biological age reducing regimen 34, thereby creating different variations of the combination of sleep quality variables 42 with different sets of values, instructing the peripheral device 20 to administer the different variations of the combination of sleep quality variables 42, deriving sets of biological age indicators 44 of the user 12 from the detected physiological activity of the user 12 respectively in response to the different variations of the combination of biological age variables 42 administered to the user 12, and optimizing the biological age reducing regimen 34 of the user 12 based on the different variations of the combination of biological age variables 42 of the biological age reducing regimen 34 and the derived sets of biological age indicators 44 of the user 12.

In still another embodiment, rather than focusing on sleep quality or the biological age of the user 12, the non-invasive self-autonomous system 10 may be specially configured for minimizing the biological energy expenditure of the brain 14 (i.e., the mental energy) of the user 12. The non-invasive self-autonomous system 10 will determine a lifestyle regimen, and in this case an efficient mental energy regimen, that minimizes the mental energy expenditure of the brain 14 of the user 12. The lifestyle optimizer 22 may take the form of a specialized mental energy expenditure optimizer.

Although the amount of biological energy expenditure of a brain cannot yet be quantified and reduced to a single number (e.g., attebytes), negative mental brain states of the user 12 may serve as a proxy for wasted biological energy expenditure (i.e., expended biological energy that does not level the user 12 to spend time and energy of more valuable tasks). Thus, the non-invasive self-autonomous system 10 may minimize the mental energy expenditure of the user 12 by minimizing the mental energy associated with negative mental brain states. The optimized mental energy regimen is considered by the mental energy expenditure optimizer 22 to be the mental energy regimen that results in no negative mental brain states (e.g., one or more of anxiety, anger, disgust, fear, contempt, and sadness) for the user 12, in which case, the lifestyle variables are mental energy expenditure variables (avoiding a person or situation, playing music at a specified time, playing a movie at a specified time, conducting a meditative session at a specified time, etc.), the lifestyle aspect of the user 12 of which the set of qualitative indicators 44 is derived is mental energy expenditure of the user 12, the derived set of qualitative indicators 44 may be mental energy expenditure indicators (e.g., of a mental brain state breakdown of the user 12), and the target set of qualitative indicators 44′ may be target mental energy expenditure indicators (e.g., less than 5% mental brain states of the user 12).

In the illustrated embodiment, the mental energy regimen is administered to the user 12 when the user 12 is awake, while the set of mental energy expenditure indicators 44 are likewise derived from physiological signals detected in the user 12 while the user 12 is awake. In one embodiment, the set of mental energy expenditure indicators 44 are high-level mental brain states of the user 12 that are derived from the low-level physiological brain state of the user 12 acquired from the physiological signals 28 output by the non-invasive brain interface assembly 16. The set of mental energy expenditure indicators 44 can be informed by the peripheral physiological signals 32 output by the peripheral sensor(s) 18. For example, the peripheral sensor(s) 18 may take the form of an optical wearable device, e.g., a wrist-worn device, such as the wearable optical device described in U.S. Provisional Application Ser. Nos. 63/134,479, 63/154,115, 63/160,995, and 63/179,080; and U.S. patent application Ser. No. 17/550,387, which have been previously incorporated herein by reference. which can measure the heart rate, heart rate variability, respiratory rate, blood pressure, blood flow, and skin conductivity, which can be correlated to the mental brain states of the user 12. Thus, the sleep quality optimizer 22 may derive, from physiological signals 28 output by the non-invasive brain interface assembly 16 and the peripheral physiological signals 32 output by the peripheral sensor(s) 18, a set of mental energy expenditure indicators 44 in the form of the existence of any negative mental brain states experienced by the user 12.

In a similar manner described above with respect to the generic lifestyle regimen 34 of the user, the mental energy expenditure optimizer 22 is configured for modifying values of the combination of mental energy expenditure variables of the mental energy regimen 34, thereby creating different variations of the combination of mental energy expenditure variables 42 with the different sets of values to the user 12, instructing the peripheral device 20 to administer the different variations of combination of mental energy 42 with the different sets of values to the user 12, deriving sets of mental energy expenditure indicators 44 of the user 12 from the detected physiological activity of the user 12 respectively in response to the different variations of the combination of mental energy expenditure variables 42 administered to the user 12, and optimizing the mental energy regimen 34 of the user 12 based on the different variations of the combination of mental energy expenditure variables 42 of the mental energy regimen 34 and the derived sets of mental energy expenditure indicators 44 of the user 12.

In an optional embodiment, the mental energy expenditure optimizer 22 is configured for instructing the peripheral device 20 to prompt the user 12 to enter the nature of an activity that the user 12 is currently performing (e.g., interacting with social media or communicating with a particular person) in response a set of mental energy expenditure indicators 44 of a negative mental brain state of the user 12 derived from currently detected physiological activity of the user 12, such that at least one of the different variations of the combination of mental energy expenditure variables 42 (corresponding to the nature of the activity currently performed by the user 12) has manually entered values of the mental energy expenditure variables. In this manner, the mental energy expenditure optimizer 22, when optimizing the mental energy regimen 34 of the user 12, may focus on those mental energy expenditure variables (in this example, interacting with social media or communicating with a particular person) that are prone to induce a negative mental brain state in the user 12. For example, the mental energy expenditure optimizer 22 may instruct the peripheral device 20 to administer an optimized or unoptimized lifestyle regimen 34 to the user 12 that includes a mental energy expenditure variable not to interact with social media (or limit interaction with social media for a period of time (e.g., one hour a day) or not to communicate with a particular person (or limit interaction with that particular person).

Referring to FIG. 5, a physical implementation of one embodiment of non-invasive self-autonomous system 110a that may embody the non-invasive self-autonomous system 10 illustrated in FIG. 1 will now be described. The non-invasive self-autonomous system 110a comprises an optically-based non-invasive brain interface assembly 116a configured for optically detecting neural activity in the brain 14 of the user 12. The non-invasive brain interface assembly 116a may, e.g., incorporate any one or more of the neural activity detection technologies described in U.S. patent application Ser. No. 15/844,370, entitled “Pulsed Ultrasound Modulated Optical Tomography Using Lock-In Camera” (now U.S. Pat. No. 10,335,036), U.S. patent application Ser. No. 15/844,398, entitled “Pulsed Ultrasound Modulated Optical Tomography With Increased Optical/Ultrasound Pulse Ratio” (now U.S. Pat. No. 10,299,682), U.S. patent application Ser. No. 15/844,411, entitled “Optical Detection System For Determining Neural Activity in Brain Based on Water Concentration” (now U.S. Pat. No. 10,420,469), U.S. patent application Ser. No. 15/853,209, entitled “System and Method For Simultaneously Detecting Phase Modulated Optical Signals” (now U.S. Pat. No. 10,016,137), U.S. patent application Ser. No. 15/853,538, entitled “Systems and Methods For Quasi-Ballistic Photon Optical Coherence Tomography In Diffusive Scattering Media Using a Lock-In Camera” (now U.S. Pat. No. 10,219,700), U.S. patent application Ser. No. 16/266,818, entitled “Ultrasound Modulating Optical Tomography Using Reduced Laser Pulse Duration,” U.S. patent application Ser. No. 16/299,067, entitled “Non-Invasive Optical Detection Systems and Methods in Highly Scattering Medium” (now U.S. Pat. No. 11,206,985), U.S. patent application Ser. No. 16/379,090, entitled “Non-Invasive Frequency Domain Optical Spectroscopy For Neural Decoding,” U.S. patent application Ser. No. 16/382,461, entitled “Non-Invasive Optical Detection System and Method,” U.S. patent application Ser. No. 16/392,963, entitled “Interferometric Frequency-Swept Source And Detector In A Photonic Integrated Circuit,” U.S. patent application Ser. No. 16/392,973, entitled “Non-Invasive Measurement System and Method Using Single-Shot Spectral-Domain Interferometric Near-Infrared Spectroscopy Based On Orthogonal Dispersion, U.S. patent application Ser. No. 16/393,002, entitled “Non-Invasive Optical Detection System and Method Of Multiple-Scattered Light With Swept Source Illumination,” U.S. patent application Ser. No. 16/385,265, entitled “Non-Invasive Optical Measurement System and Method for Neural Decoding” (now U.S. Pat. No. 11,096,585), U.S. patent application Ser. No. 16/533,133, entitled “Time-Of-Flight Optical Measurement And Decoding Of Fast-Optical Signals,” U.S. patent application Ser. No. 16/565,326, entitled “Detection Of Fast-Neural Signal Using Depth-Resolved Spectroscopy,” U.S. patent application Ser. No. 16/226,625, entitled “Spatial and Temporal-Based Diffusive Correlation Spectroscopy Systems and Methods” (now U.S. Pat. No. 10,420,498), U.S. Provisional Application Ser. No. 62/772,584, entitled “Diffuse Correlation Spectroscopy Measurement Systems and Methods,” U.S. patent application Ser. No. 16/432,793, entitled “Non-Invasive Measurement Systems with Single-Photon Counting Camera” (now U.S. Pat. No. 11,213,206) U.S. patent application Ser. No. 16/842,443, entitled “Interferometric Parallel Detection Using Digital Rectification and Integration” (now U.S. Pat. No. 11,060,843), U.S. patent application Ser. No. 16/842,488, entitled “Interferometric Parallel Detection Using Analog Data Compression” (now U.S. Pat. No. 11,119,039) and U.S. patent application Ser. No. 16/842,523, entitled “Partially Balanced Interferometric Parallel Detection,” which are all expressly incorporated herein by reference.

The brain interface assembly 116a includes a wearable unit 124a configured for being applied to the user 12, and in this case, worn on the head of the user 12; and an auxiliary head-worn or non-head-worn unit 126a (e.g., worn on the neck, shoulders, chest, or arm). Alternatively, the functionality of the non-head-worn unit 126a may be incorporated into the head-worn unit 124a. The auxiliary non-head-worn unit 126a may be coupled to the head-worn unit 124a via a wired connection 128 (e.g., electrical wires). Alternatively, the brain interface assembly 116a may use a non-wired connection (e.g., wireless radio frequency (RF) signals (e.g., Bluetooth, Wifi, cellular, etc.) or optical links (e.g., fiber optic or infrared (IR)) for providing power to or communicating between the respective head-worn unit 124a and the auxiliary unit 126a.

The head-worn unit 124a comprises electronic or optical components, such as, e.g., one or more optical sources, an interferometer, one or more optical detector(s) (not shown), etc., an output port 130a for emitting sample light 132 generated by the brain interface assembly 116a into the head of the user 12, an input port 130b configured for receiving neural-encoded signal light 134 from the head of the user 12, which signal light is then detected, modulated and/or processed to determine brain activity of the user 12, and a support housing structure 136 containing the electronic or optical components, and ports 130a, 130b.

The support housing structure 136 may be shaped, e.g., have a banana, headband, cap, helmet, beanie, other hat shape, or other shape adjustable and conformable to the user's head, such that the ports 130a, 130b are in close contact with the outer skin of the head, and in this case, the scalp of the user 12. The support housing structure 136 may be made out of any suitable cloth, soft polymer, plastic, hard shell, and/or any other suitable material as may serve a particular implementation. In an alternative embodiment, optical fibers (not shown) may be respectively extended from the ports 130a, 130b, thereby freeing up the requirement that the ports 130a, 130b be disposed in close proximity to the surface of the head. In any event, an index matching fluid may be used to reduce reflection of the light generated by the head-worn unit 124a from the outer skin of the scalp. An adhesive, strap, or belt (not shown) can be used to secure the support housing structure 136 to the head of the user 12.

The auxiliary unit 126a comprises a housing 138 containing a controller 140 and a processor 142. The controller 140 is configured for controlling the operational functions of the head-worn unit 124a, whereas the processor 142 is configured for processing the neural-encoded signal light 134 acquired by the head-worn unit 124a to detect and localize the brain activity of the user 12.

The auxiliary unit 126a may additionally include a power supply (which if head-worn, may take the form of a rechargeable or non-chargeable battery), a control panel with input/output functions, a display, and memory. Alternatively, power may be provided to the auxiliary unit 126a wirelessly (e.g., by induction).

In the illustrated embodiment, the functionality of the lifestyle optimizer 22 illustrated in FIG. 1 is also performed by the auxiliary unit 126a. For example, the controller 140 and processor 142 may respectively correspond to the controller 60 and processor 62 of the lifestyle optimizer 22 illustrated in FIG. 4, with the controller 140 configured for instructing a peripheral device 120 (described in further detail below) to administer the lifestyle regimen 34 containing a combination of lifestyle variables to the user 12, and the processor 142 configured for selecting or modifying the values of the combination of lifestyle variables, such that the peripheral device 120 administers the different variations of the combination of lifestyle variables 42 of the lifestyle regimen 34, deriving the different sets of qualitative indicators 44 of the lifestyle aspect of the user 12 from the detected physiological activity of the user 12 (e.g., the physiological signals 28 characterizing the brain activity 26 of the user 12 output by the non-invasive brain interface assembly 110a and/or the physiological signals 32 characterizing the peripheral physiological activity 30 of the user 12 output by the peripheral sensor(s) 118 (described in further detail below)) respectively in response to the different variations of the combination of lifestyle variables 42 of the lifestyle regimen 34 administered to the user 12 by the peripheral device 120. In alternative embodiments, at least a portion of the functionality of the lifestyle optimizer 22 may be performed in other components, such as the peripheral device 120.

The functionality of the peripheral sensor(s) 118 is similar to the functionality of the peripheral sensor(s) 18 illustrated in FIG. 1. In the illustrated embodiment, the peripheral sensor(s) 118 take the form of a wrist-worn device, such as, a Whoop strap, Fitbit, Garmin, Apple Watch, etc., or the form of wearable time-domain optical device described in U.S. Provisional Application Ser. Nos. 63/134,479, 63/154,115, 63/160,995, and 63/179,080; and U.S. patent application Ser. No. 17/550,387, which have been previously incorporated herein by reference. The peripheral sensor(s) 118 are coupled to the brain interface assembly 116a (and in this case, to the auxiliary unit 126a) via a wired connection 144 (e.g., electrical wires). Alternatively, a non-wired connection (e.g., wireless radio frequency (RF) signals (e.g., Bluetooth, Wifi, cellular, etc.) or optical links (e.g., fiber optic or infrared (IR)) for providing power to or communicating between the respective the auxiliary unit 126a of the brain interface assembly 116a and the peripheral sensor(s) 118 may be used.

The functionality of the peripheral device 120 is similar to the functionality of the peripheral device 20 illustrated in FIG. 1. The peripheral device 120 (e.g., a Smartphone, tablet computer, or the like) that contains the functionality of the computer 16, although in alternative embodiments, at least some of the processing functions of computer 16 can be performed in other processing components, such as the processor 142 of the auxiliary unit 126a. The peripheral device 120 is coupled to the auxiliary unit 126a of the brain interface assembly 116a via a wireless connection 146 (e.g., wireless radio frequency (RF) signals (e.g., Bluetooth, Wifi, cellular, etc.) or optical links (e.g., fiber optic or infrared (IR)) for communicating between the peripheral device 120 and the brain interface assembly 116a. Alternatively, wired connections between the peripheral device 120 and the brain interface assembly 116a may be used.

The non-invasive self-autonomous system 110a further comprises a database, server, or cloud structure 124, the functionality of which is similar to the functionality of the database, server, or cloud structure 24 illustrated in FIG. 1. The database, server, or cloud structure 124 may be coupled to the auxiliary unit 126a of the brain interface assembly 116a (and/or the peripheral device 120) via a wireless connection 148 (e.g., wireless radio frequency (RF) signals (e.g., Bluetooth, Wifi, cellular, etc.) or optical links (e.g., fiber optic or infrared (IR)) for communicating between the database, server, or cloud structure 124 and the brain interface assembly 116a and peripheral device 120. Alternatively, a wired connection between the database, server, or cloud structure 124 and the auxiliary unit 126a of the brain interface assembly 116a and/or the peripheral device 120 may be used in the case where additional or supplemental computational power is required.

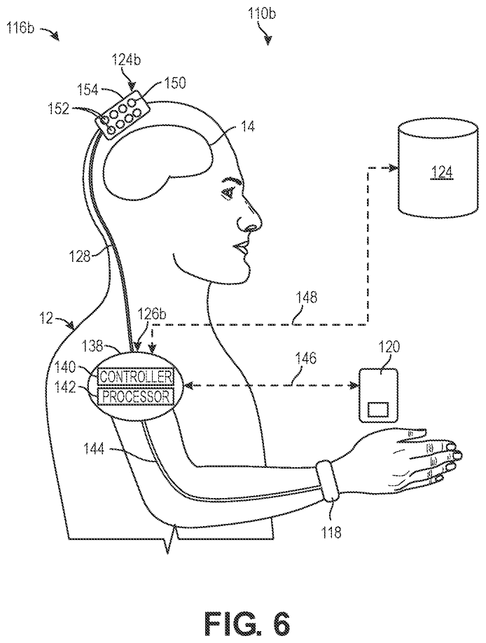

Referring to FIG. 6, a physical implementation of another embodiment of a non-invasive self-autonomous system 110b that may embody the non-invasive self-autonomous system 10 illustrated in FIG. 1 will now be described.

The non-invasive self-autonomous system 110b comprises an optically-based, time-domain, non-invasive brain interface assembly 116b configured for optically detecting neural activity in the brain 14 of the user 12. Example time domain-based optical measurement techniques include, but are not limited to, time-correlated single-photon counting (TCSPC), time domain near infrared spectroscopy (TD-NIRS), time domain diffusive correlation spectroscopy (TD-DCS), and time domain Digital Optical Tomography (TD-DOT). The non-invasive brain interface assembly 116b may, e.g., incorporate any one or more of the neural activity detection technologies described in U.S. Non-Provisional application Ser. No. 16/051,462, entitled “Fast-Gated Photodetector Architecture Comprising Dual Voltage Sources with a Switch Configuration” (now U.S. Pat. No. 10,158,038), U.S. patent application Ser. No. 16/202,771, entitled “Non-Invasive Wearable Brain Interface Systems Including a Headgear and a Plurality of Self-Contained Photodetector Units Configured to Removably Attach to the Headgear” (now U.S. Pat. No. 10,340,408), U.S. patent application Ser. No. 16/177,351, entitled “Photodetector Comprising a Single Photon Avalanche Diode and a Capacitor” (now U.S. Pat. No. 10,424,683), U.S. patent application Ser. No. 16/177,351, entitled “Wearable Systems with Fast-Gated Photodetector Architectures Having a Single Photon Avalanche Diode and Capacitor” (now U.S. Pat. No. 10,672,936), U.S. patent application Ser. No. 16/177,351, entitled “Non-Invasive Wearable Brain Interface Systems Including a Headgear and a Plurality of Self-Contained Photodetector Units” (now U.S. Pat. No. 10,672,935), U.S. patent application Ser. No. 16/856,524, entitled “Wearable Brain Interface Systems Including a Headgear and a Plurality Of Photodetector Units” (now U.S. Pat. No. 11,004,998), U.S. patent application Ser. No. 17/213,664, entitled “Wearable Brain Interface Systems Including a Headgear and a Plurality Of Photodetector Units,” U.S. patent application Ser. No. 16/283,730, entitled “Stacked Photodetector Assemblies” (now U.S. Pat. No. 10,515,993), U.S. patent application Ser. No. 16/544,850, entitled “Wearable Systems with Stacked Photodetector Assemblies” (now U.S. Pat. No. 10,847,563), U.S. patent application Ser. No. 16/844,860, entitled “Photodetector Architectures for Time-Correlated Single Photon Counting,” U.S. patent application Ser. No. 16/852,183, entitled “Photodetector Architectures for Efficient Fast-Gating Comprising a Control System Controlling a Current Drawn by an Array of Photodetectors with a Single Photon Avalanche Diode” (now U.S. Pat. No. 11,081,611), U.S. patent application Ser. No. 16/880,686, entitled “Photodetector Systems with Low-Power Time-To-Digital Converter Architectures” (now U.S. Pat. No. 10,868,207), U.S. patent application Ser. No. 17/202,554, entitled “Control Circuit for a Light Source in an Optical Measurement System,” U.S. patent application Ser. No. 17/176,307, entitled “Multiplexing Techniques for Interference Reduction in Time-Correlated Signal Photon Counting,” U.S. patent application Ser. No. 17/202,563, entitled “Maintaining Consistent Photodetector Sensitivity in an Optical Measurement System,” U.S. patent application Ser. No. 17/202,572, entitled “Phase Lock Loop Circuit Based Adjustment of a Measurement Time Window in an Optical Measurement System,” U.S. patent application Ser. No. 17/202,583, entitled “Techniques for Determining a Timing Uncertainty of a Component of an Optical Measurement System,” U.S. patent application Ser. No. 17/202,588, entitled “Techniques for Characterizing a Nonlinearity of a Time-To-Digital Converter in an Optical Measurement System,” U.S. patent application Ser. No. 17/202,598, entitled “Temporal Resolution Control for Temporal Point Spread Function Generation in an Optical Measurement System,” U.S. patent application Ser. No. 17/202,613, entitled “Bias Voltage Generation in an Optical Measurement System,” U.S. patent application Ser. No. 17/202,631, entitled “Detection of Motion Artifacts in Signals Output by Detectors of a Wearable Optical Measurement System,” U.S. patent application Ser. No. 17/202,651, entitled “Dynamic Range Optimization in an Optical Measurement System,” U.S. patent application Ser. No. 17/202,657, entitled “Maintaining Consistent Photodetector Sensitivity in an Optical Measurement System,” U.S. patent application Ser. No. 17/202,668, entitled “Photodetector Calibration of an Optical Measurement System,” U.S. patent application Ser. No. 17/176,448, entitled “Estimation of Source-Detector Separation in an Optical Measurement System,” U.S. patent application Ser. No. 17/176,460, entitled “Wearable Module Assemblies for an Optical Measurement System” (now U.S. Pat. No. 11,096,620), U.S. patent application Ser. No. 17/176,466, entitled “Wearable Devices and Wearable Assemblies with Adjustable Positioning for Use in an Optical Measurement System,” U.S. patent application Ser. No. 17/176,470, entitled “Integrated Detector Assemblies for a Wearable Module of an Optical Measurement System,” U.S. patent application Ser. No. 17/176,487, entitled “Detector Assemblies for a Wearable Module of an Optical Measurement System and Including Spring-Loaded Light-Receiving Members,” U.S. patent application Ser. No. 17/176,539, entitled “Integrated Light Source Assembly with Laser Coupling for a Wearable Optical Measurement System,” U.S. patent application Ser. No. 17/176,309, entitled “Multimodal Wearable Measurement Systems and Methods,” U.S. patent application Ser. No. 17/176,560, entitled “Time Domain-Based Optical Measurement System and Method Configured to Measure Absolute Properties of Tissue,” U.S. patent application Ser. No. 17/233,033, entitled “Systems and Methods for Noise Removal in an Optical Measurement System,” U.S. patent application Ser. No. 17/324,819, entitled “Systems and Methods for Data Representation in an Optical Measurement System,” U.S. patent application Ser. No. 17/176,315, entitled “Systems, Circuits, and Methods for Reducing Common-Mode Noise in Biopotential Recordings,” and Han Y. Ban, et al., “Kernel Flow: A High Channel Count Scalable TD-fNIRS System,” SPIE Photonics West Conference (Mar. 6, 2021), which are all expressly incorporated herein by reference.

The brain interface assembly 116b includes a head-worn unit 124b that is configured for being applied to the user 12, and in this case, worn on the head of the user 12; and an auxiliary non-head-worn unit 126b (e.g., worn on the neck, shoulders, chest, or arm). Alternatively, the functionality of the non-head-worn unit 126b may be incorporated into the head-worn unit 124b, as described below. The auxiliary non-head-worn unit 126b may be coupled to the head-worn unit 124b via a wired connection 128 (e.g., electrical wires). Alternatively, the brain interface assembly 116b may use a non-wired connection (e.g., wireless radio frequency (RF) signals (e.g., Bluetooth, Wifi, cellular, etc.) or optical links (e.g., fiber optic or infrared (IR)) for providing power to or communicating between the respective head-worn unit 124b and the auxiliary unit 126b.

The head-worn unit 124b includes one or more light sources 150 configured for generating light pulses. The light source(s) 150 may be configured for generating one or more light pulses at one or more wavelengths that may be applied to a desired target (e.g., a target within the brain). The light source(s) 150 may be implemented by any suitable combination of components. For example, light source(s) 150 described herein may be implemented by any suitable device. For example, a light source as used herein may be, for example, a distributed feedback (DFB) laser, a super luminescent diode (SLD), a light emitting diode (LED), a diode-pumped solid-state (DPSS) laser, a laser diode (LD), a super luminescent light emitting diode (sLED), a vertical-cavity surface-emitting laser (VCSEL), a titanium sapphire laser, a micro light emitting diode (mLED), and/or any other suitable laser or light source.

The head-worn unit 124b includes a plurality of photodetector units 152, e.g., comprising single-photon avalanche diodes (SPADs) configured for detecting a single photon (i.e., a single particle of optical energy) in each of the light pulses. For example, an array of these sensitive photodetector units can record photons that reflect off of tissue within the brain in response to application of one or more of the light pulses generated by the light sources 150. Based on the time it takes for the photons to be detected by the photodetector units, neural activity and other attributes of the brain can be determined or inferred. Each photodetector unit 152 includes a plurality of individual photodetectors. Any number of photodetectors may be employed (e.g., 256, 512, . . . , 26384, etc.), where n is an integer greater than or equal to one (e.g., 4, 5, 8, 20, 21, 24, etc.). The photodetectors may be arranged in any suitable manner and each may each be implemented by any suitable circuit configured to detect individual photons of light incident upon the photodetectors.

Photodetector units that employ the properties of a SPAD are capable of capturing individual photons with very high time-of-arrival resolution (a few tens of picoseconds). When photons are absorbed by a SPAD, their energy frees bound charge carriers (electrons and holes) that then become free-carrier pairs. In the presence of an electric field created by a reverse bias voltage applied to the diode, these free-carriers are accelerated through a region of the SPAD, referred to as the multiplication region. As the free carriers travel through the multiplication region, they collide with other carriers bound in the atomic lattice of the semiconductor, thereby generating more free carriers through a process called impact ionization. These new free-carriers also become accelerated by the applied electric field and generate yet more free-carriers. This avalanche event can be detected and used to determine an arrival time of the photon. In order to enable detection of a single photon, a SPAD is biased with a reverse bias voltage having a magnitude greater than the magnitude of its breakdown voltage, which is the bias level above which free-carrier generation can become self-sustaining and result in a runaway avalanche. This biasing of the SPAD is referred to as arming the device. When the SPAD is armed, a single free carrier pair created by the absorption of a single photon can create a runaway avalanche resulting in an easily detectable macroscopic current. The SPAD may be gated in any suitable manner or be configured to operate in a free running mode with passive quenching. For example, the photodetectors may be configured to operate in a free-running mode, such that photodetectors are not actively armed and disarmed (e.g., at the end of each predetermined gated time window). In contrast, while operating in the free-running mode, photodetectors may be configured to reset within a configurable time period after an occurrence of a photon detection event (i.e., after a photodetector detects a photon) and immediately begin detecting new photons. However, only photons detected within a desired time window (e.g., during each gated time window) may be included in the histogram that represents a light pulse response of the target (e.g., a temporal point spread function (TPSF)). The terms histogram and TPSF are used interchangeably herein to refer to a light pulse response of a target, e.g., the brain of the user 12.

It will be recognized that in some alternative embodiments, the head-worn unit 124b may include a single light source 150 and/or single photodetector unit 152. For example, the brain interface assembly 116b may be used for controlling a single optical path and for transforming photodetector pixel measurements into an intensity value that represents an optical property of a brain tissue region. In some alternative embodiments, the head-worn unit 124b does not include individual light sources. Instead, a light source configured to generate the light that is detected by the photodetector may be included elsewhere in the brain interface assembly 116b. For example, a light source may be included in the auxiliary unit 126b. In alternative embodiments, a module assembly may house the photodetector units 152 and the light source 150 in the same assembly and eliminate the need for long fiber optic cables. For example, the head-worn unit 124b may include the wearable modular assembly wherein the wearable modular assembly includes a plurality of connectable wearable modules. Each wearable module includes a light source 150 configured to emit a light pulse toward a target within the brain of the user and a plurality of photodetector units 152 configured to receive photons included in the light pulse after the photons are scattered by the target. The wearable module assemblies can conform to a 3D surface of the user's head, maintain tight contact of the detectors with the user's head to prevent detection of ambient light, and maintain uniform and fixed spacing between light sources 150 and photodetector units 152. The wearable module assemblies may also accommodate a large variety of head sizes, from a young child's head size to an adult head size, and may accommodate a variety of head shapes and underlying cortical morphologies through the conformability and scalability of the wearable module assemblies. These exemplary modular assemblies and systems are described in more detail in U.S. patent application Ser. Nos. 17/176,470; 17/176,487; 17/176,539; 17/176,560; 17/176,460; and Ser. No. 17/176,466, which have been previously incorporated herein by reference.

The head-worn unit 124b further comprises a support housing structure 154 containing the light source(s) 150, photodetector units 152, and other electronic or optical components. In alternative embodiments, the housing structure 154 may include a single module assembly containing a single light source 150, plurality of photodetector units 152, and other electronic or optical components. In other alternative embodiments, the housing structure 154 may include a plurality of module assemblies tiled together, wherein each module assembly includes the light source 150, plurality of photodetector units 152, and other electronic or optical components. As will be described in further detail below, the support housing structure 154 may be shaped, e.g., have a banana, headband, cap, helmet, beanie, other hat shape, or other shape adjustable and conformable to the user's head, such that the photodetector units 152 are in close contact with the outer skin of the head, and in this case, the scalp of the user 12. The support housing structure 154 may be made out of any suitable cloth, soft polymer, plastic, hard shell, and/or any other suitable material as may serve a particular implementation.

While the brain interface assembly 116b shows one head-worn unit 124b, any suitable number of head-worn units 124b may be used, for instance at different locations on the head.

The auxiliary unit 126b comprises the housing 138 containing the controller 140 and the processor 142. The controller 140 is configured for controlling the operational functions of the head-worn unit 124b, whereas the processor 142 is configured for processing the photons acquired by the head-worn unit 124b to detect and localize the detected neural activity 24 of the user 12. The auxiliary unit 126b may additionally include a power supply (which if head-worn, may take the form of a rechargeable or non-chargeable battery), a control panel with input/output functions, a display, and memory. Alternatively, power may be provided to the auxiliary unit 126b wirelessly (e.g., by induction). In the same manner described above with respect to the non-invasive self-autonomous system 110a, the functionality of the lifestyle optimizer 22 illustrated in FIG. 1 is performed by the auxiliary unit 126b, e.g., by the controller 140 and processor 142.

The non-invasive self-autonomous system 110b further comprises the peripheral device 120, peripheral sensor(s) 118, and database, server, or cloud structure 124, which can function and be coupled to each other and the non-invasive brain assembly 114b in the same manner described above with respect to the non-invasive self-autonomous system 110a.

As another example, the non-invasive brain interface assembly 16 may be implemented by a wearable multimodal measurement system configured to perform both optical-based brain data acquisition operations and electrical-based brain data acquisition operations, such as any of the wearable multimodal measurement systems described in U.S. patent application Ser. Nos. 17/176,315 and 17/176,309, which have been previously incorporated herein by reference.

The wearable multimodal measurement systems may at least partially implement optical measurement system described in Han, et al., and as described in the optically-based U.S. Patent Applications that have been previously incorporated herein by reference, which include a wearable assembly with N light sources, M detectors, but also includes X electrodes. N, M, and X may each be any suitable value (i.e., there may be any number of light sources, any number of detectors, and any number of electrodes in the non-invasive brain interface assembly 16 as may serve a particular implementation). The electrodes may be configured to detect electrical activity within a target (e.g., the brain of the user 12). Such electrical activity may include electroencephalogram (EEG) activity and/or any other suitable type of electrical activity as may serve a particular implementation. In some examples, the electrodes are all conductively coupled to one another to create a single channel that may be used to detect electrical activity. Alternatively, at least one electrode included in the X number of electrodes is conductively isolated from a remaining number of electrodes to create at least two channels that may be used to detect electrical activity.

Such optically-based systems described herein employ a time domain-based (e.g., TD-NIRS) measurement technique) and may detect blood oxygenation levels and/or blood volume levels by measuring the change in shape of laser pulses after they have passed through target tissue, e.g., the brain of the user 12. As used herein, a shape of laser pulses refers to a temporal shape, as represented for example by a histogram generated by a time-to-digital converter (TDC) coupled to an output of the photodetector.

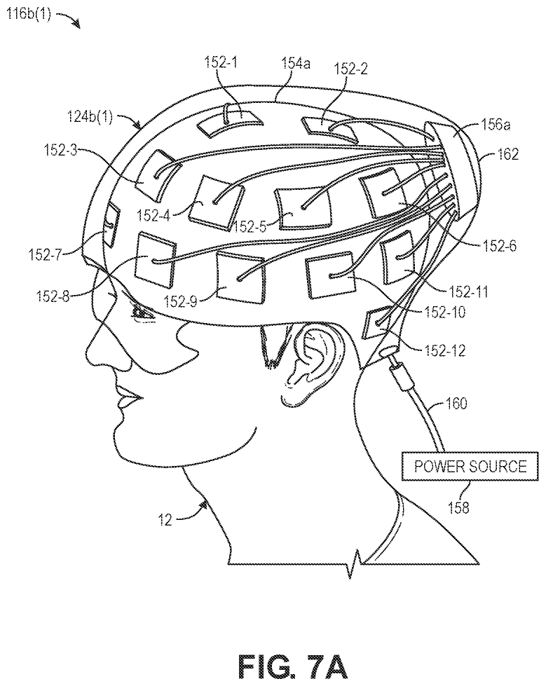

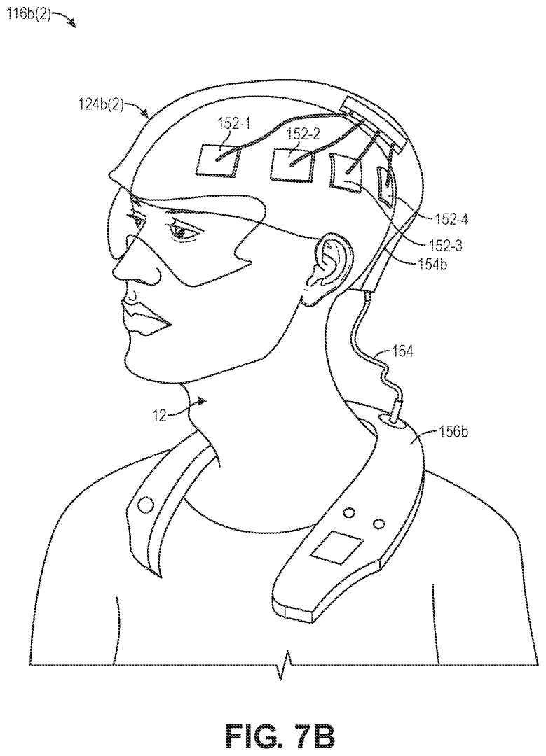

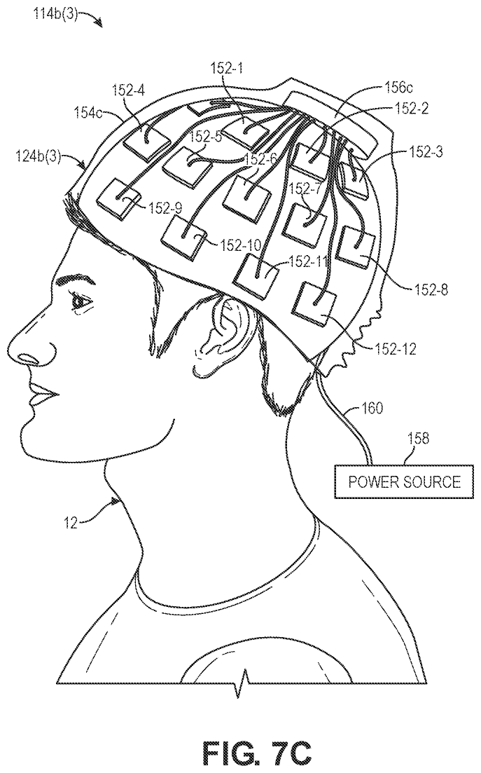

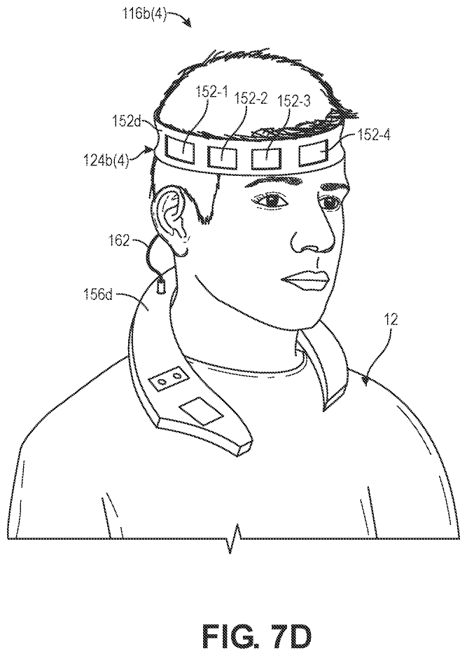

Referring now to FIGS. 7A-7D, different embodiments of the brain interface assembly 116b will be described. Such brain interface assemblies 116b may communicate wirelessly or via wire with the peripheral device 120 and database, server, cloud structure 124, as described above.

Each of the brain interface assemblies 116b described below comprises a head-worn unit 124b having a plurality of photodetector units 152 and a support housing structure 154 in which the photodetector units 152 are embedded within individual slots or cut-outs. Each of the photodetector units 152 may comprise, e.g., a SPAD, voltage sources, capacitors, switches, and any other circuit components and other optical components (not shown) required to detect photons. Each of the brain interface assemblies 116b may also comprise one or more light sources (not shown) for generating light pulses, although the source of such light may be derived from ambient light in some cases. In alternative embodiments, the light source may be a component contained within of the photodetector units. Each of brain interface assemblies 116b may also comprise a control/processing unit 156, such as, e.g., a control circuit, time-to-digital (TDC) converter, and signal processing circuit for controlling the operational functions of the photodetector units 152 and any light source(s), and processing the photons acquired by photodetector units 152 to detect and localize the brain activity of the user 12. As will be described in further detail below, the control/processing unit 156 may be contained in the head-worn unit 124b or may be incorporated into a self-contained auxiliary unit. As will be set forth below, the support housing structure 154 may be shaped, e.g., have a banana, headband, cap, helmet, beanie, other hat shape, or other shape adjustable and conformable to the user's head, such that the photodetector units 152 are in close contact with the outer skin of the head, and in this case, the scalp of the user 12.

As shown in FIG. 7A, a brain interface assembly 116b(1) comprises a head-worn unit 124b(1) and a power source 158 coupled to the head-worn unit 124b(1) via a power cord 160. The head-worn unit 124b(1) includes the photodetector units 152 (shown as 152-1 through 152-12) and a control/processing unit 156a. The head-worn unit 124b(1) further includes a support housing structure 154a that takes a form of a cap that contains the photodetector units 152 and control/processing unit 156a. The material for the cap 154a may be selected out of any suitable cloth, soft polymer, plastic, hard shell, and/or any other suitable material as may serve a particular implementation. The power source 158 may be implemented by a battery and/or any other type of power source configured to provide operating power to the photodetector units 152, control/processing unit 156a, and any other component included within the brain interface assembly 116b(1) via the power cord 160. The head-worn unit 124b(1) optionally includes a crest or other protrusion 162 formed in the cap 154a for providing means of carrying/housing the control/processing unit 156a.

As shown in FIG. 7B, a brain interface assembly 116b(2) comprises a head-worn unit 124b(2) and a control/processing unit 156b coupled to the head-worn unit 124b(2) via a wired connection 164. The head-worn unit 124b(2) includes the photodetector units 152 (shown as 152-1 through 152-4), and a support housing structure 154b that takes the form of a helmet containing the photodetector units 152. The material for the helmet 154b may be selected out of any suitable polymer, plastic, hard shell, and/or any other suitable material as may serve a particular implementation. Unlike the control/processing unit 156a of the brain interface assembly 116b(1) illustrated in FIG. 7A, which is contained in the head-worn unit 124b(1), the control/processing unit 156b is self-contained, and may take the form of a garment (e.g., a vest, partial vest, or harness) for being worn on the shoulders of the user 12. The self-contained control/processing unit 156b may additionally include a power supply (which if head-worn, may take the form of a rechargeable or non-chargeable battery), a control panel with input/output functions, a display, and memory. Alternatively, power may be provided to the self-contained control/processing unit 156b wirelessly (e.g., by induction).

As shown in FIG. 7C, a brain interface assembly 116b(3) comprises a head-worn unit 124b(3) and a power source 158 coupled to the head-worn unit 124b(3) via a power cord 160. The head-worn unit 124b(3) includes the photodetector units 152 (shown as 152-1 through 152-12) and a control/processing unit 156c. The head-worn unit 124b(3) further includes a support housing structure 154c that takes a form of a beanie that contains the photodetector units 152 and control/processing unit 156c. The material for the beanie 154c may be selected out of any suitable cloth, soft polymer, plastic, and/or any other suitable material as may serve a particular implementation. The power source 158 may be implemented by a battery and/or any other type of power source configured to provide operating power to the photodetector units 152, control/processing unit 156c, and any other component included within the brain interface assembly 116b(3) via a wired connection 160.

As shown in FIG. 7D, a brain interface assembly 116b(4) comprises a head-worn unit 124b(4) and a control/processing unit 156d coupled to the head-worn unit 124b(4) via a wired connection 164. The head-worn unit 124b(4) includes the photodetector units 152 (shown as 152-1 through 152-4), and a support housing structure 154d that takes a form of a headband containing the photodetector units 152. The material for the headband 154d may be selected out of any suitable cloth, soft polymer, plastic, hard shell, and/or any other suitable material as may serve a particular implementation. The control/processing unit 156d is self-contained, and may take the form of a garment (e.g., a vest, partial vest, or harness) for being worn on the shoulders of the user 12. The self-contained control/processing unit 156d may additionally include a power supply (which if head-worn, may take the form of a rechargeable or non-chargeable battery), a control panel with input/output functions, a display, and memory. Alternatively, power may be provided to the self-contained control/processing unit 156d wirelessly (e.g., by induction).

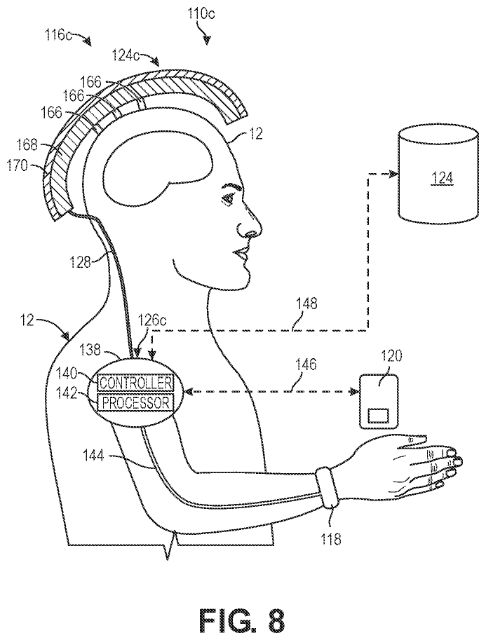

Referring to FIG. 8, a physical implementation of still another embodiment of a system 110c that may embody the non-invasive self-autonomous system 10 illustrated in FIG. 1 will now be described.

The non-invasive self-autonomous system 110c comprises a magnetically-based non-invasive brain interface assembly 116c configured for magnetically detecting neural activity in the brain 14 of the user 12. Example techniques of using the magnetically-based non-invasive brain interface assembly 116c are directed to the area of magnetic field measurement systems including systems for magnetoencephalography (MEG). The non-invasive brain interface assembly 116c may, e.g., incorporate any one or more of the neural activity detection technologies described in U.S. patent application Ser. No. 16/428,871, entitled “Magnetic Field Measurement Systems and Methods of Making and Using,” U.S. patent application Ser. No. 16/418,478, entitled “Magnetic Field Measurement System and Method of Using Variable Dynamic Range Optical Magnetometers” (now U.S. Pat. No. 10,976,386), U.S. patent application Ser. No. 16/418,500, entitled, “Integrated Gas Cell and Optical Components for Atomic Magnetometry and Methods for Making and Using,” U.S. patent application Ser. No. 16/457,655, entitled “Magnetic Field Shaping Components for Magnetic Field Measurement Systems and Methods for Making and Using” (now U.S. Pat. No. 10,983,177), U.S. patent application Ser. No. 16/213,980, entitled “Systems and Methods Including Multi-Mode Operation of Optically Pumped Magnetometer(S),” (now U.S. Pat. No. 10,627,460), U.S. patent application Ser. No. 16/814,926, entitled “Systems and Methods Including Multi-Mode Operation of Optically Pumped Magnetometer(S)” (now U.S. Pat. No. 10,877,111), U.S. patent application Ser. No. 17/105,338, entitled “Systems and Methods Including Multi-Mode Operation of Optically Pumped Magnetometer(S),” U.S. patent application Ser. No. 16/456,975, entitled “Dynamic Magnetic Shielding and Beamforming Using Ferrofluid for Compact Magnetoencephalography (MEG),” U.S. patent application Ser. No. 16/752,393, entitled “Neural Feedback Loop Filters for Enhanced Dynamic Range Magnetoencephalography (MEG) Systems and Methods” (now U.S. Pat. No. 11,022,658), U.S. patent application Ser. No. 16/741,593, entitled “Magnetic Field Measurement System with Amplitude-Selective Magnetic Shield,” U.S. patent application Ser. No. 16/820,131, entitled “Integrated Magnetometer Arrays for Magnetoencephalography (MEG) Detection Systems and Methods,” U.S. patent application Ser. No. 16/850,380, entitled “Systems and Methods for Suppression of Interferences in Magnetoencephalography (MEG) and Other Magnetometer Measurements,” U.S. patent application Ser. No. 16/850,444, entitled “Compact Optically Pumped Magnetometers with Pump and Probe Configuration and Systems and Methods,” U.S. Provisional Application Ser. No. 62/842,818, entitled “Active Shield Arrays for Magnetoencephalography (MEG),” U.S. patent application Ser. No. 16/928,810, entitled “Systems and Methods for Frequency and Wide-Band Tagging of Magnetoencephalography (MEG) Signals,” U.S. patent application Ser. No. 16/984,720, entitled “Systems and Methods for Multiplexed or Interleaved Operation of Magnetometers,” U.S. patent application Ser. No. 16/984,752, entitled “Systems and Methods having an Optical Magnetometer Array with Beam Splitters” (now U.S. Pat. No. 10,996,293), U.S. patent application Ser. No. 17/004,507, entitled “Methods and Systems for Fast Field Zeroing for Magnetoencephalography (MEG),” U.S. patent application Ser. No. 16/862,826, entitled “Single Controller for Wearable Sensor Unit that Includes an Array Of Magnetometers” (now U.S. Pat. No. 11,131,723), U.S. patent application Ser. No. 16/862,856, entitled “Systems and Methods for Measuring Current Output By a Photodetector of a Wearable Sensor Unit that Includes One or More Magnetometers” (now U.S. Pat. No. 11,131,724), U.S. patent application Ser. No. 16/862,879, entitled “Interface Configurations for a Wearable Sensor Unit that Includes One or More Magnetometers” (now U.S. Pat. No. 11,131,725), U.S. patent application Ser. No. 16/862,901, entitled “Systems and Methods for Concentrating Alkali Metal Within a Vapor Cell of a Magnetometer Away from a Transit Path of Light,” U.S. patent application Ser. No. 16/862,919, entitled “Magnetic Field Generator for a Magnetic Field Measurement System,” U.S. patent application Ser. No. 16/862,946, entitled “Magnetic Field Generator for a Magnetic Field Measurement System,” U.S. patent application Ser. No. 16/862,973, entitled “Magnetic Field Measurement Systems Including a Plurality of Wearable Sensor Units Having a Magnetic Field Generator,” U.S. patent application Ser. No. 17/160,078, entitled “Self-Calibration of Flux Gate Offset and Gain Drift To Improve Measurement Accuracy of Magnetic Fields from the Brain Using a Wearable Neural Detection System,” U.S. patent application Ser. No. 17/160,109, entitled “Nested and Parallel Feedback Control Loops for Ultra-Fine Measurements of Magnetic Fields from the Brain Using a Neural Detection System,” U.S. patent application Ser. No. 17/160,152, entitled “Estimating the Magnetic Field at Distances from Direct Measurements to Enable Fine Sensors to Measure the Magnetic Field from the Brain Using a Neural Detection System,” U.S. patent application Ser. No. 17/160,179, entitled “Systems and Methods that Exploit Maxwell's Equations and Geometry to Reduce Noise For Ultra-Fine Measurements of Magnetic Fields from the Brain Using a Neural Detection System,” U.S. patent application Ser. No. 17/160,195, entitled “Optimal Methods to Feedback Control and Estimate Magnetic Fields to Enable a Neural Detection System to Measure Magnetic Fields from the Brain,” U.S. patent application Ser. No. 17/328,235, entitled “Systems and Methods for Recording Neural Activity,” U.S. patent application Ser. No. 17/338,429, entitled “OPM Module Assembly with Alignment and Mounting Components as Used in a Variety of Headgear Arrangements,” U.S. patent application Ser. No. 17/328,271, entitled “Systems and Methods for Multimodal Pose and Motion Tracking for Magnetic Field Measurement Or Recording Systems,” U.S. patent application Ser. No. 17/328,290, entitled “Magnetic Field Measurement or Recording Systems with Validation Using Optical Tracking Data,” U.S. patent application Ser. No. 17/569,287, entitled “Devices, Systems, Methods with Optical Pumping Magnetometers for Three-Axis Magnetic Field Sensing,” U.S. patent application Ser. No. 17/572,404, entitled “Devices, Systems, and Methods with a Piezoelectric-Driven Light Intensity Modulator,” U.S. Provisional Application Ser. No. 63/224,768, entitled “Devices, Systems, and Methods for Suppressing Optical Noise in Optically Pumped Magnetometers,” and Ethan J. Pratt, et al., “Kernel Flux: A Whole-Head 432-Magnetometer Optically-Pumped Magnetoencephalography (OP-MEG) System for Brain Activity Imaging During Natural Human Experiences,” SPIE Photonics West Conference (Mar. 6, 2021), which are all expressly incorporated herein by reference.

The brain interface assembly 116c includes a magnetoencephalography (MEG) head-worn unit 124c that is configured for being applied to the user 12, and in this case, worn on the head of the user 12; and an auxiliary non-head-worn unit 126c (e.g., worn on the neck, shoulders, chest, or arm). Alternatively, the functionality of the non-head-worn unit 126c may be incorporated into the head-worn unit 124c, as described below. The auxiliary non-head-worn unit 126c may be coupled to the head-worn unit 124c via a wired connection 128 (e.g., electrical wires). Alternatively, the brain interface assembly 116c may use a non-wired connection (e.g., wireless radio frequency (RF) signals (e.g., Bluetooth, Wifi, cellular, etc.) or optical links (e.g., fiber optic or infrared (IR)) for providing power to or communicating between the respective head-worn unit 124c and the auxiliary unit 126c.

The head-worn unit 124c includes a plurality of optically pumped magnetometers (OPMs) 166 or other suitable magnetometers to measure biologically generated magnetic fields from the brain of the user 12 and a passive shield 168 (and/or flux concentrators). By placing the passive shield 168 over the head of the user 12, the ambient background magnetic field arising from areas outside the passive shield 168 is greatly decreased and the OPMs 166 can measure or detect magnetic fields from activity occurring in the brain of the user 12 due to the reduction in the ambient background magnetic field.

An OPM is used in an optical magnetometry system used to detect a magnetic field that propagates through the human head. Optical magnetometry can include the use of optical methods to measure a magnetic field with very high accuracy—on the order of 1×10⁻¹⁵ Tesla. Of particular interest for their high-sensitivity, an OPM can be used in optical magnetometry to measure weak magnetic fields. (The Earth's magnetic field is typically around 50 micro Tesla). In at least some systems, the OPM has an alkali vapor gas cell that contains alkali metal atoms in a combination of gas, liquid, or solid states (depending on temperature). The gas cell may contain a quenching gas, buffer gas, or specialized anti-relaxation coatings or any combination thereof. The size of the gas cells can vary from a fraction of a millimeter up to several centimeters, allowing the practicality of OPMs to be used with wearable non-invasive brain interface devices.

The head-worn unit 124c further comprises a support housing structure 170 containing the OPMs 166, passive shield 168, and other electronic or magnetic components. As will be described in further detail below, the support housing structure 170 may be shaped, e.g., have a banana, headband, cap, helmet, beanie, other hat shape, or other shape adjustable and conformable to the user's head, such that the OPMs 166 are in close contact with the outer skin of the head, and in this case, the scalp of the user 12. The support housing structure 170 may be made out of any suitable cloth, soft polymer, plastic, hard shell, and/or any other suitable material as may serve a particular implementation.

The head-worn unit 124c may also include a plurality of optically pumped magnetometer (OPM) modular assemblies, which OPM modular assemblies are enclosed within the head-worn unit 124c. The OPM modular assembly is designed to enclose the elements of the OPM optics, vapor cell, and detectors in a compact arrangement that can be positioned close to the head of the human subject. The head-worn unit 124c may also include an adjustment mechanism used for adjusting the head-worn unit 124c to conform with the human subject's head. These exemplary OPM modular assemblies and systems are described in more detail in U.S. patent application Ser. No. 17/338,429, which has been previously incorporated by reference. The magnetically-based head-worn unit 124c can also be used in a magnetically shielded environment with an open entryway which can allow for user movement as described for example in U.S. patent application Ser. No. 17/328,235, which has been previously incorporated herein by reference. User tracking movement in a magnetically shielded environment can include an optical user pose identification system and/or other sensing modalities as described more fully in U.S. patent application Ser. Nos. 17/328,271 and 17/328,290, which have been previously incorporated herein by reference.

The auxiliary unit 126c comprises the housing 138 containing the controller 140 and the processor 142. The controller 140 is configured for controlling the operational functions of the head-worn unit 124c, whereas the processor 142 is configured for processing the magnetic fields detected by the head-worn unit 124c to detect and localize the detected neural activity 24 of the user 12. The auxiliary unit 126c may additionally include a power supply (which if head-worn, may take the form of a rechargeable or non-chargeable battery), a control panel with input/output functions, a display, and memory. Alternatively, power may be provided to the auxiliary unit 126c wirelessly (e.g., by induction). In the same manner described above with respect to the non-invasive self-autonomous system 110a, the functionality of the lifestyle optimizer 22 illustrated in FIG. 1 is performed by the auxiliary unit 126c, e.g., by the controller 140 and processor 142.

The non-invasive self-autonomous system 110c further comprises the peripheral device 120, peripheral sensor(s) 118, and database, server, or cloud structure 124, which can function and be coupled to each other and the non-invasive brain assembly 114c in the same manner described above with respect to the non-invasive self-autonomous system 110a.

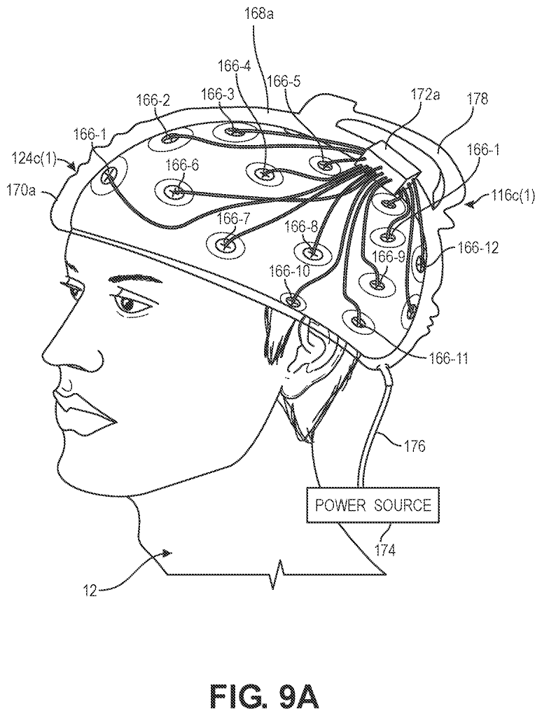

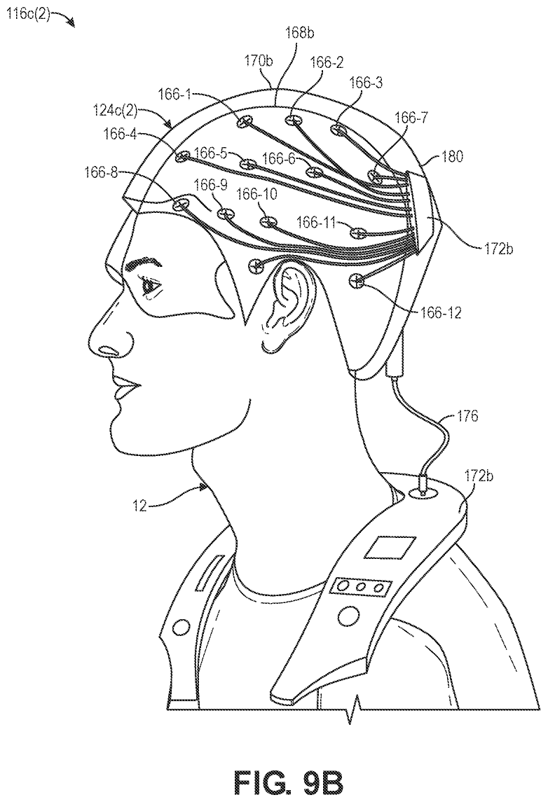

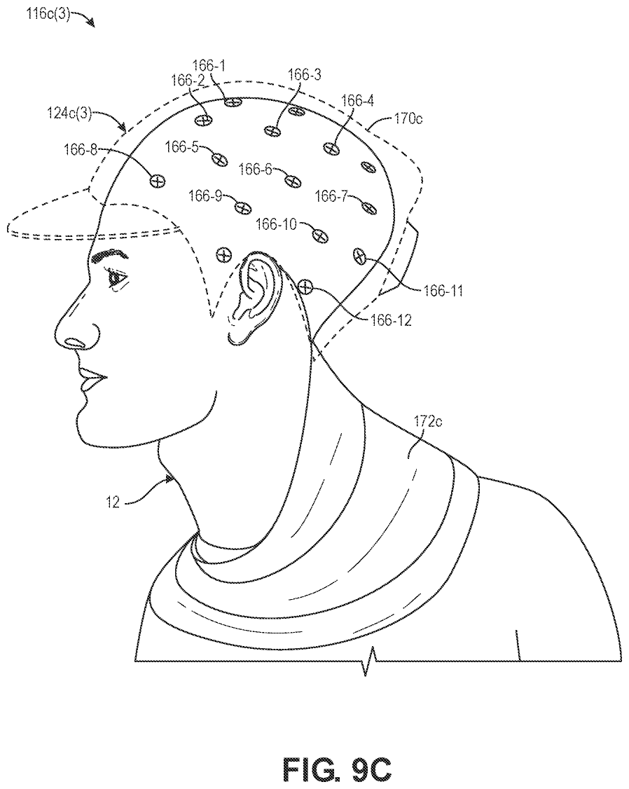

Referring now to FIGS. 9A-9C, different embodiments of the brain interface assembly 116c will be described. Such brain interface assemblies 116c may communicate wirelessly or via wire with the peripheral device 120 and the database, server, cloud structure 124, as described above. Each of the brain interface assemblies 116c described below comprises a head-worn unit 124c having a plurality of OPMs 166, a passive shield 168, and a support housing structure 170 in which the OPMs 166 and passive shield 168 are embedded. Each of brain interface assemblies 116c may also comprise a control/processing unit 172 for controlling the operational functions of the OPMs 166, and processing the magnetic fields detected by the OPMs 166 to detect and localize the brain activity of the user 12. As will be described in further detail below, the control/processing unit 172 may be contained in the head-worn unit 124c or may be incorporated into a self-contained auxiliary unit. As will be set forth below, the support housing structure 170 may be shaped, e.g., have a banana, headband, cap, helmet, beanie, other hat shape, or other shape adjustable and conformable to the user's head, such that the OPMs 166 are in close contact with the outer skin of the head, and in this case, the scalp of the user 12.

As shown in FIG. 9A, a brain interface assembly 116c(1) comprises a head-worn unit 124c(1) and a power source 174 coupled to the head-worn unit 124c(1) via a wired connection 176. The head-worn unit 124c(1) includes the OPMs 166 (shown as 166-1 through 166-12) and a control/processing unit 172a. The head-worn unit 124c(1) further includes a support housing structure 170a that takes a form of a helmet that contains the OPMs 166, passive shield 168, and control/processing unit 172a. The material for the helmet 170a may be selected out of any suitable cloth, soft polymer, plastic, hard shell, and/or any other suitable material as may serve a particular implementation. The power source 174 may be implemented by a battery and/or any other type of power source configured to provide operating power to the OPMs 166, control/processing unit 172a, and any other component included within the brain interface assembly 116c(1) via the wired connection 176. The head-worn unit 124c(1) optionally includes a handle 178 affixed to the helmet 170a for providing a convenient means of carrying the head-worn unit 124c(1).

As shown in FIG. 9B, a brain interface assembly 116c(2) comprises a head-worn unit 124c(2) and a control/processing unit 172b coupled to the head-worn unit 124b(2) via a wired connection 176. The head-worn unit 124c(2) includes the OPMs 166 (shown as 166-1 through 166-12), and a support housing structure 170b that takes a form of a helmet that contains the OPMs 166 and passive shield 168. The material for the helmet 170b may be selected out of any suitable cloth, soft polymer, plastic, hard shell, and/or any other suitable material as may serve a particular implementation. Unlike the control/processing unit 172a of the brain interface assembly 116c(1) illustrated in FIG. 9A, which is contained in the head-worn unit 124c(1), the control/processing unit 172b is self-contained, and may take the form of a garment (e.g., a vest, partial vest, or harness) for being worn on the shoulders of the user 12. The self-contained control/processing unit 172b may additionally include a power supply (which if head-worn, may take the form of a rechargeable or non-chargeable battery), a control panel with input/output functions, a display, and memory. Alternatively, power may be provided to the self-contained control/processing unit 172b wirelessly (e.g., by induction). The head-worn unit 124c(1) optionally includes a crest or other protrusion 180 formed in the helmet 170b for providing means of carrying a control/processing unit 172b′.

As shown in FIG. 9C, a brain interface assembly 116c(3) comprises a head-worn unit 124c(3) and a control/processing unit 172c. The head-worn unit 124c(3) includes the OPMs 166 (shown as 166-1 through 166-12), and a support housing structure 170c that takes a form of a baseball cap that contains the OPMs 166 and passive shield 168. The material for baseball cap 170c may be selected out of any suitable cloth, soft polymer, plastic, hard shell, and/or any other suitable material as may serve a particular implementation. The control/processing unit 172c is self-contained, and may take the form of a garment (e.g., scarf) for being worn around the neck of the user 12. The self-contained control/processing unit 172c may additionally include a power supply (which if head-worn, may take the form of a rechargeable or non-chargeable battery), a control panel with input/output functions, a display, and memory. Alternatively, power may be provided to the self-contained control/processing unit 172c wirelessly (e.g., by induction).

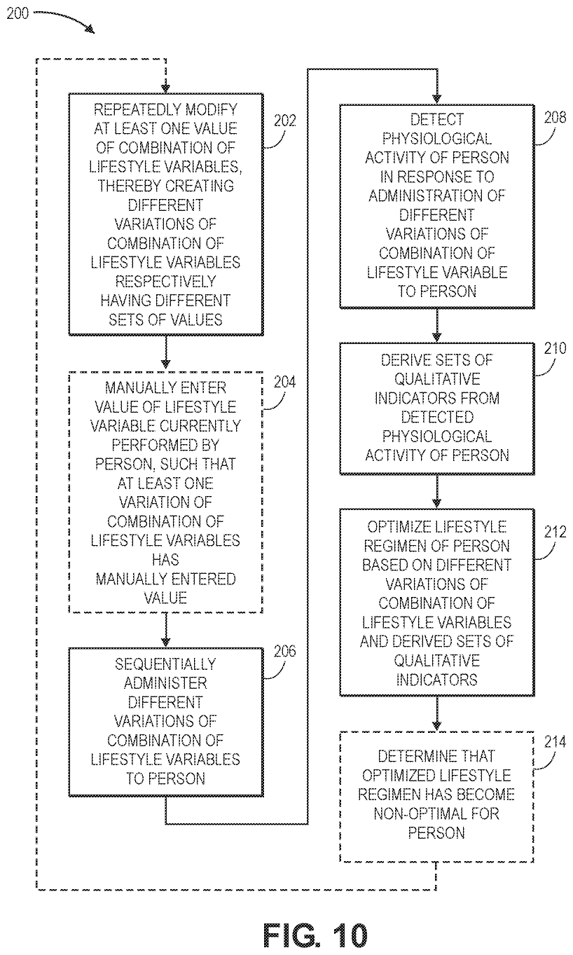

Having described the structure and function of the non-invasive self-autonomous system 10, one method 200 of operating the non-invasive self-autonomous system 10 to optimize a lifestyle regimen 34 containing a combination of lifestyle variables of a user 12 (i.e., a person) will now be described with reference to FIG. 10. The lifestyle regimen 34 of the person 12 to be optimized may be, e.g., a sleep quality regimen or a mental energy expenditure regimen, and the lifestyle aspect of the person 12 may be, e.g., a sleep quality of the person 12 or a mental energy expenditure of the person 12.

First, at least one value of the combination of lifestyle variables 42 (e.g., sleep quality variables or mental energy expenditure variables) is repeatedly modified, thereby creating different variations of the combination of lifestyle variables 42 respectively having different sets of values (e.g., via the lifestyle optimizer 22) (step 202). In an optional method, the person 12 is allowed to manually enter a value of a lifestyle variable currently performed by the person 12 (e.g., in response to a prompt to enter a manually entered value), such that at least one variation of the combination of lifestyle variables 42 has the manually entered value (step 204).

Next, the different variations of the combination of lifestyle variables 42 are sequentially administered (e.g., on a daily basis) to the person 12 (e.g., via instructions from the lifestyle optimizer 22 to the peripheral device 20) (step 206). Then, physiological activity of the person 12 is detected (e.g., brain activity 26 of the person 12 by the non-invasive brain interface assembly 16 and/or the peripheral physiological activity 30 of the person 12 is detected by the peripheral sensor(s) 18) in response to the administration of the different variations of the combination of lifestyle variables 42 to the person 12 (step 208).

Next, sets of qualitative indicators 44 of the lifestyle aspect (e.g., sleep quality indicators or mental energy expenditure variables) of the person 12 are derived from the detected physiological activity of the person 12 (step 210). Lastly, the lifestyle regimen 34 of the person 12 is optimized based on the different variations of the combination of lifestyle variables 42 and the derived sets of quality indicators 44 (e.g., via the lifestyle optimizer 22) (step 212). For example, one of the different variations of the combination of lifestyle variables 42 may be selected for the optimized lifestyle regimen 34. In an optional method, the optimized lifestyle regimen 34 may be subsequently determined to become non-optimal for the person 12 (e.g., via the lifestyle optimizer 22) (step 214). In this case, steps 202-212 are repeated, such that the lifestyle regimen 34 of the person 12 is re-optimized.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. A non-invasive self-autonomous system, comprising:
a peripheral device configured for administering a lifestyle regimen containing a combination of lifestyle variables to a user;
at least one non-invasive measurement device configured for detecting physiological activity of the user in response to the administration of the lifestyle regimen to the user; and
a lifestyle optimizer configured for modifying values of the combination of lifestyle variables of the lifestyle regimen, such that the peripheral device sequentially administers different variations of the combination of lifestyle variables respectively having different sets of values to the user, the lifestyle optimizer further configured for deriving sets of qualitative indicators of an aspect of a lifestyle of the user from the detected physiological activity of the user respectively, and optimizing the lifestyle regimen of the user based on the different variations of the combination of lifestyle variables and the derived sets of qualitative indicators.

2. The non-invasive self-autonomous system of claim 1, wherein the lifestyle optimizer is configured for selecting one of the different variations of the combination of lifestyle variables for the optimized lifestyle regimen.

3. The non-invasive self-autonomous system of claim 1, wherein the lifestyle optimizer is configured for determining that the optimized lifestyle regimen has become non-optimal for the user, modifying values of the combination of lifestyle variables of the lifestyle regimen again, such that the peripheral device sequentially administers other different variations of the combination of lifestyle variables respectively having other different sets of values to the user, deriving other sets of qualitative indicators of the lifestyle aspect of the user from the detected physiological activity of the user respectively in response to the other different variations of combination of lifestyle variables sequentially administered to the user, and reoptimizing the lifestyle regimen of the user based on the other different variations of the combination of lifestyle variables and the other derived sets of qualitative indicators.

4. The non-invasive self-autonomous system of claim 1, wherein the peripheral device is configured for allowing the user to manually enter a value of a lifestyle variable, such that at least one variation of the combination of lifestyle variables has the manually entered value.

5. The non-invasive self-autonomous system of claim 4, wherein the value of the lifestyle variable is currently performed by the user, and the lifestyle optimizer is configured for instructing the peripheral device to prompt the user to manually enter the currently performed value of the lifestyle variable.

6. The non-invasive self-autonomous system of claim 1, wherein the lifestyle optimizer is a sleep quality optimizer, the lifestyle regimen is a sleep quality regimen, the lifestyle variables are sleep quality variables, the qualitative indicators are sleep quality indicators, and the lifestyle aspect is sleep of the user.

7. The non-invasive self-autonomous system of claim 6, wherein the sleep quality variables comprise at least one of going to bed at a specified time, waking up at a specified time, not drinking alcohol or caffeine after a specified time, not eating food after a specified time, utilizing blue light blocking glasses and deep-wave sound machine, performing an exercise routine at a specified time, stop working at a specified time, reading a book or watching a movie, meditation or breathwork, setting bedroom at a certain temperature at bedtime.

8. The non-invasive self-autonomous system of claim 6, wherein the at least one non-invasive measurement device is configured for detecting the physiological activity of the user while the user is sleeping, and the peripheral device is configured for sequentially administering the different variations of the combination of sleep quality variables to the user while the user is awake.

9. The non-invasive self-autonomous system of claim 6, wherein each derived set of sleep quality indicators comprises a percentage breakdown between Light Sleep, Deep Sleep, REM, and Awake of the user.

10. The non-invasive self-autonomous system of claim 6, wherein each derived set of sleep quality indicators comprises an amount of Deep Sleep of the user.

11. The non-invasive self-autonomous system of claim 1, wherein the lifestyle optimizer is a biological age optimizer, the lifestyle regimen is a biological age regimen, the lifestyle variables are biological age variables, the qualitative indicators are biological age indicators, and the lifestyle aspect is biological age of the user.

12. The non-invasive self-autonomous system of claim 11, wherein the qualitative indicators comprise epigenic data.

13. The non-invasive self-autonomous system of claim 12, wherein the epigenic data comprises a pattern of DNA methylation of a genome of the user.

14. The non-invasive self-autonomous system of claim 11, wherein the qualitative indicators comprise brain data of the user.

15. The non-invasive self-autonomous system of claim 14, wherein the at least one non-invasive measurement device comprises a non-invasive brain interface assembly, the detected physiological activity of the user comprises detected brain activity of the user, and each derived set of qualitative indicators comprises a physiological brain state of the user comprising the brain data.

16. The non-invasive self-autonomous system of claim 11, wherein the biological age indicators are biological age indicators of different organs of the user, and the biological age of the user comprises biological ages of different organs of the user.

17. The non-invasive self-autonomous system of claim 1, wherein the lifestyle optimizer is a mental energy expenditure optimizer, the lifestyle regimen is an efficient mental energy regimen, the lifestyle variables are mental energy expenditure variables, the qualitative indicators are mental energy expenditure indicators, and the lifestyle aspect of the user is mental energy expenditure of the user.

18. The non-invasive self-autonomous system of claim 17, wherein the mental energy expenditure variables comprise at least one of avoiding a person or situation, playing music at a specified time, playing a movie at a specified time, and conducting a meditative session at a specified time.

19. The non-invasive self-autonomous system of claim 17, wherein the at least one non-invasive measurement device is configured for detecting the physiological activity of the user while the user is awake, and the peripheral device is configured for sequentially administering the different variations of the combination of mental energy expenditure variables to the user while the user is awake.

20. The non-invasive self-autonomous system of claim 17, wherein each derived set of mental energy expenditure indicators comprises emotional brain states of the user.

21. The non-invasive self-autonomous system of claim 17, wherein the mental energy expenditure optimizer is configured for prompting the user to manually enter a value of a mental energy expenditure variable currently performed by the user, such that at least one variation of the combination of mental energy expenditure variables has the manually entered value.

22. The non-invasive self-autonomous system of claim 1, wherein the at least one non-invasive measurement device comprises a non-invasive brain interface assembly, the detected physiological activity of the user comprises detected brain activity of the user, and each derived set of qualitative indicators comprises a brain state of the user.

23. The non-invasive self-autonomous system of claim 22, wherein the brain state of the user is a physiological brain state of the user.

24. The non-invasive self-autonomous system of claim 22, wherein the brain state of the user is a mental brain state of the user.

25. The non-invasive self-autonomous system of claim 22, wherein the non-invasive brain interface assembly is an optical measurement assembly.

26. (canceled)

27. The non-invasive self-autonomous system of claim 22, wherein the non-invasive brain interface assembly comprises at least one detector configured for detecting energy from a brain of the user, and processing circuitry configured for identifying the brain activity in response to detecting the energy from the brain of the user.

28. The non-invasive self-autonomous system of claim 27, wherein the non-invasive brain interface assembly comprises a head-worn unit carrying the at least one energy source.

29. The non-invasive self-autonomous system of claim 27, wherein the non-invasive brain interface assembly comprises an auxiliary non-head-worn unit carrying the processing circuitry.

30. The non-invasive self-autonomous system of claim 1, wherein the at least one non-invasive measurement device comprises one or more peripheral sensors, and the detected physiological activity of the user comprises detected peripheral physiological activity.

31. The non-invasive self-autonomous system of claim 30, wherein the peripheral physiological activity comprises at least one of heart rate, heart rate variability, respiratory rate, blood pressure, blood flow, skin conductivity, blood glucose, Cortisol, DHEA-s, Serotonin, GABA, Dopamine, Norepinephrine, Epinephrine, and Glutamate.

32.-69. (canceled)
